SEQUENCE LISTING (24 sequences)

SEQ ID NO: 1 - protein, 15 aa, Peptide Binder of HPV E6
LLVITIWQLWDEMLS

SEQ ID NO: 2 - protein, 15 aa, Peptide Binder of HPV E6
IDYSTAMNLLDSLLS

SEQ ID NO: 3 - protein, 15 aa, Peptide Binder of HPV E6
QTMNHALDVLYCLLG

SEQ ID NO: 4 - protein, 14 aa, Peptide Binder of HPV E6
ILRICLTCWIGFSS

SEQ ID NO: 5 - protein, 15 aa, Peptide Binder of HPV E6
LSKGAFILLDMLLGA

SEQ ID NO: 6 - protein, 17 aa, Peptide Binder of HPV E6
HDNFLELALEVLDPSLE

SEQ ID NO: 7 - protein, 15 aa, Peptide Binder of HPV E6
TLGWVTVFERLLGCD

SEQ ID NO: 8 - protein, 9 aa, Peptide Binder of HPV E6
  misc_feature (9)..(9): Xaa = unknown or other
DADVPSNPX

SEQ ID NO: 9 - protein, 15 aa, Peptide Binder of HPV E6
  misc_feature (6)..(6): Xaa = unknown or other
FCNAAXASNNSLXXX

SEQ ID NO: 10 - protein, 15 aa, Peptide Binder of HPV E6
APDLWYEIWDIILGK

SEQ ID NO: 11 - protein, 16 aa, Peptide Binder of HPV E6
KCCLCALLALFPPPSE

SEQ ID NO: 12 - protein, 15 aa, Peptide Binder of HPV E6
ADLHCCCLTCRIEAL

SEQ ID NO: 13 - DNA, 31 bp, OVT 3302
ccccggatcccgggccgaggcggccccgcgg

SEQ ID NO: 14 - DNA, 32 bp, OVT 3349
cccccctcgaggcggccgcggccattatggcc

SEQ ID NO: 15 - DNA, 13 bp, Aptamer 3305
cggccacgctgga

SEQ ID NO: 16 - DNA, 76 bp, Aptamer 3307
  misc_feature (17)..(18): n = A or T or G or C
tggcctatttattcavnnvnnvnnvnnvnnvnnvnnvnnvnnvnnvnnvnnvnnvnnvnntccagcgtggccgcct

SEQ ID NO: 17 - DNA, 18 bp, Aptamer 3352
tgaataaataggccataa

SEQ ID NO: 18 - DNA, 76 bp, OVT 3307
  misc_feature (17)..(18): n = A or T or G or C
tccgccggtgcgacctnnvnnvnnvnnvnnvnnvnnvnnvnnvnnvnnvnnvnnvnnvnnvacttatttatccggt

SEQ ID NO: 19 - DNA, 44 bp, OVT3391
ggggggaattcttatacttgtgtttctctgcgtcgttggagtcg

SEQ ID NO: 20 - DNA, 40 bp, OVT 3390
gggggcatatgatggcgcgctttgaggatccaacacggcg

SEQ ID NO: 21 - DNA, 32 bp, OVT 1510
taatcttctgcttccatttcttctcttccctc

SEQ ID NO: 22 - DNA, 28 bp, OVT 1561
ggcggaaggagggggcggcggggggcgg

SEQ ID NO: 23 - DNA, 42 bp, OVT 1513
ggggggatcctaatcttctgcttccatttcttctcttccctc

SEQ ID NO: 24 - DNA, 45 bp, OVT 1514
ggggaagcttcgcgggggtggccggggccagggcttcccacggtg

(19) United States
(12) Patent Application Publication
(10) Pub. No.: US 2002/0045188 A1
(43) Pub. Date: Apr. 18, 2002

(54) Methods for validating polypeptide targets that correlate to cellular phenotypes

(76) Inventors: Carl Alexander Kamb, Salt Lake City, UT (US); Giordano Michael Caponigro, Salt Lake City, UT (US); David Heng-Fai Teng, Salt Lake City, UT (US); Tanya Marie Sandrock, Salt Lake City, UT (US); Mark Stump, Salt Lake City, UT (US)

Correspondence Address: Li-Hsien Rin-Laures, MD, MARSHALL, O'TOOLE, GERSTEIN, MURRAY & BORUN, 6300 Sears Tower, 233 South Wacker Drive, Chicago, IL, 60606-6402, US

(21) Appl. No.: 09/865,644

(22) Filed: May 25, 2001

Related U.S. Application Data
(63) Continuation-in-part of application No. 09/193,759, filed on Nov. 17, 1998, now abandoned.
(63) Continuation-in-part of application No. PCT/US99/27409, filed on Apr. 10, 2001.

(51) Int. Cl. G01N 33/53; G01N 33/567; C12N 15/63

(52) U.S. Cl. 435/7.1; 435/7.21; 435/455

(57) ABSTRACT

Generally applicable methods for identifying physiologically relevant endogenous target molecules, are provided. The methods use both protein interaction assay steps and phenotypic assay steps. In some embodiments, protein interactions are detected utilizing yeast two hybrid techniques.

### RELATED APPLICATIONS

This application is a continuation-in-part from priority document U.S. Ser. No. [not yet received], filed Apr. 10, 2001, (“Methods for Validating Polypeptide Targets that Correlate to Cellular Phenotypes”) which is the national phase entry of corresponding PCT application US99/27419 (same title), and is a CIP of priority document U.S. Ser. No. 09/193,759 (same title). All three of the prior related applications are specifically incorporated by reference in their entireties.

### FIELD OF THE INVENTION

The present invention comprises generally applicable methods for identifying endogenous, physiologically relevant cellular components, often endogenous proteins or polypeptides, that are involved in cellular pathways correlating to a phenotype of interest. These cellular components may be readily identified and/or validated through their interactions with exogenous agents or probes, often “perturbagens,” and are preferably characterized by an ability to bind more than one independent, physiologically relevant perturbagen. Physiological relevance is assessed by use of a bioassay. By use of these methods, potential therapeutic agents are subjected to parallel validation, and physiologically irrelevant false positives can be readily eliminated.

### BACKGROUND

Most drug development schemes require accurate identification of the endogenous components of physiological pathways that can lead to disease-for example, to cancer. These endogenous components may be potential therapeutic targets, or may point the way to genes that are associated with occurrence of the disease. Identification of such physiologically relevant components (i.e., components that participate in a cellular pathway of interest), however, has been time-consuming and uncertain.

Various protein-protein, protein/DNA, protein/RNA or enzyme-substrate interactions outside, on or within the cell (“endogenous cellular interactions”) may be of particular interest because these interactions provide a means for identifying molecular mechanisms and physiologically relevant components that underlie a disorder or disease state in an organism. For example, once one relevant endogenous cellular interaction is identified, it may be explored in more depth, often enabling the associated physiologically relevant genes and/or cellular pathways to be identified. In addition, a physiologically relevant endogenous cellular interaction provides the basis for screening potential therapeutic agents. It is critical that such endogenous cellular interactions be identified accurately, so that resources are not expended pursuing interactions that ultimately are not physiologically relevant in the target cell.

Using perturbagens to identify relevant endogenous interactions offers advantages in streamlining the identification of physiologically relevant endogenous cellular interactions. Perturbagens often are proteinaceous molecules that interact with endogenous proteins in a cell, and either partially or completely disrupt the normal function of an endogenous cellular pathway. This disruption of specific biochemical interactions generates a correlative “mutant” phenotype, which may in turn be used as a selection characteristic. Perturbagens include proteinaceous moieties (peptides, polypeptides or proteins), nucleic acids, or other compounds.

Even with the advantages of using perturbagens to identify endogenous proteinaceous components, a variety of difficulties inhere in linking any detectable proteinaceous component to the actual physiological pathways in the target cell via specific binding interactions. For example, current systems for detecting protein-ligand or enzyme-substrate interactions often detect false positive results of at least two varieties: (1) interactions that are spurious artifacts of the assay system used to detect the protein-ligand interactions, and which do not reflect bonafide interactions in the endogenous environment of the cell under study (termed herein, “artifactual interactions”), and (2) interactions that do occur in the endogenous cellular environment, but which are not relevant to the cellular pathway of interest (termed herein, “non-relevant interactions”). Conversely, current assay methodologies also provide undesirable false negatives, in which physiologically relevant interactions (interactions relevant to the cellular pathway of interest) evade detection. Moreover, when the sensitivity of an assay is increased so as to decrease false negatives, more false positives may result.

Two general methods are most commonly used to assay for protein-ligand interactions—biochemical methods, and quasi—genetic methods. Both suffer from technical drawbacks.

The biochemical approach is typified by affinity purification techniques that are well known to those of skill in the art. Briefly, affinity purification techniques use a selected protein or peptide as an affinity reagent, which is brought into contact with a reaction mixture. Components that interact with that affinity reagent are then isolated and purified. This general method is of limited utility when the interaction between the target and reagent is not stable or strong, or when proteases that digest one or both of the binding partners are present in the reaction mixture. Moreover, this method undesirably can produce false positives and false negatives, in which a physiologically relevant binding partner that occurs in very low concentrations is not detected due to the presence of more abundant, yet less specifically-bound or strongly interacting proteins. Those proteins are false positives that can compete with the true positive for binding with the affinity reagent and thus mask the presence of the true positive.

The quasi-genetic approach is exemplified by a technique known to those of skill in the art as the two-hybrid assay. E.g., The yeast two-hybrid system, Oxford Univ. Press (1997), Bartel, Paul L. and Fields, Stanley, Ed. This assay often is performed in yeast cells (although it can be adapted for use in mammalian and bacterial cells), and relies upon constructing a first vector having an interaction probe or “bait” that typically is fused to a DNA binding domain (“BD”) moiety, and a second vector having an interaction target or “prey” that typically is fused to a DNA transcriptional moiety (the “activation domain” or “AD”). When the bait and prey interact, the AD and BD moieties are brought into sufficient physical proximity to result in transcription of a reporter gene (e.g., the His3 gene) located downstream of the bound complex. Prey/bait interactions are then detected by identifying yeast cells that are expressing the reporter gene—e.g., which are able to grow in the absence of histidine.

Although the yeast two-hybrid assay system is commonly used to detect protein-ligand interactions, it is known that the assay system produces false positives of several varieties. For example, in some situations the BD fusion moiety of the assay may “self-activate,” thus causing transcription of the downstream reporter gene even though there has not been a prior binding event between the BD-associated bait and the AD-associated prey (one example of an “artifactual interaction”). In other situations, the bait and prey do interact in the assay and consequently trigger transcription of the marker gene. However, the interaction between prey and bait is physiologically irrelevant because, e.g., the interaction either does not occur in vivo in the therapeutic target cell (e.g., the host cell used in the phenotypic assay) or does not play a role in the physiological pathway relevant to the phenotype under study in the therapeutic target cell (a “non-relevant interaction”).

The yeast two-hybrid technique can be adapted for high throughput protocols. Specifically, this screening technique can be adapted for the management of large sample numbers with minimal handling, in theory permitting rapid and efficient isolation of putative binding partners. This very advantage of the two-hybrid technique, however, disadvantageously magnifies the number of putative interactions from which false positives (both artifactual interactions and non-relevant interactions) must be winnowed by time-consuming individual assays or secondary screening steps.

Researchers have attempted to mitigate the false positive problem in yeast two-hybrid assays, but to date such work has focused largely on the first source of false positives—artifactual interactions (i.e., putative binding events that appear to occur in the yeast assay system but which do not occur in the endogenous cellular environment of the target cell). Such artifacts arise from a variety of factors, including oversensitivity of the yeast assay system, presence of “sticky” proteins that evidence nonspecific interactions with random molecules, self-activating molecules, and transcriptional moieties that bind DNA even absent an interaction with a second protein-binding moiety. Approaches to mitigating these artifacts include: (1) replica plating of candidate binding partners (fused to the activation domain or “AD”) with a variety of test fusion proteins on the binding domain (“BD”) moiety, with subsequent elimination of binding partners that interact with other test fusions; (2) modifying the vectors that contain the prey and bait (e.g., Louvet 0. et al., Biotechniques 23(5):816-18, 820 (1997)); (3) re-engineering the host yeast cells used in the assay (e.g., Feilotter, H E et al., Nucleic Acids Res. 22(8): 1502-3 (1994)); and (4) coimmunization and colocalization with an epitope-tagged protein (Wong, C. and Naumovski, L., Anal. Biochem. 252(1):33-39 (1997)). An approach utilizing dominant negative phenotypes to confirm interrelation of known gene products in yeast cells also has been described. (He and Jacobson, Genes Dev. 9(4):437-54 (1995)).

None of the prior art methods provide an effective, generally applicable method for improving the speed and accuracy of protein interaction screening. For example, approaches that eliminate artifactual interactions (e.g., replica plating) may be quite time-consuming and laborious, do not cull out physiologically non-relevant interactions, and may even eliminate some true positives. Moreover, even the use of a perturbagen as one component of a protein interaction assay does not preclude detection of binding events that ultimately are found to be unrelated to an endogenous pathway of interest.

Accordingly, an unmet need exists for reducing or eliminating physiologically irrelevant false-positives from protein-ligand interaction assays, thus streamlining the drug discovery process. Preferably, any solutions to this problem should be compatible with high-throughput screening techniques.

### SUMMARY OF THE INVENTION

The present invention provides methods for screening for physiologically relevant intermolecular interactions. These interactions often are between an endogenous protein or other proteinaceous molecule (referred to herein as an “endogenous protein”) and one or more corresponding ligands. Such endogenous protein-ligand interactions often participate in or indirectly affect an endogenous cellular pathway of interest. Such physiologically relevant protein-ligand interactions are detected and validated by using two independent phenotypic probes to identify and eliminate non-relevant interactions, or by interaction with a single probe when the identity of the endogenous protein as a candidate target was previously known. The methods are particularly valuable for assays involving endogenous mammalian proteins, and for streamlining and focusing high-throughput screening procedures.

The inventive methods screen for physiologically relevant protein interactions by utilizing more than one independent phenotypic probe to detect candidate targets and/or eliminate false positives therefrom. The inventive methods do so by (i) detecting the interaction between an endogenous cellular component and a primary phenotypic probe, when necessary to identify the candidate targets in the first instance, and (ii) determining whether the previously identified endogenous cellular component (the “putative therapeutic target molecule”) interacts with a second, independent phenotypic probe that provides confirmation of the physiological relevance of the target. The interactions between probes and endogenous cellular components may be detected using standard protein-ligand interaction assays—e.g., the yeast two-hybrid technology. A probe is considered “phenotypic” when it is shown in a bioassay to interact with an endogenous cellular component of the host cell and thereby cause an alteration in the same (or closely related) “phenotype of interest.” The phenotype of interest, in turn, is a detectable cellular characteristic that is an indicator of the state of an endogenous genetic pathway within a cell (e.g., a biochemical/physiological pathway that provides cell-type or cell-state specific indices such as cell growth/arrest, cell metabolic state, or cellular expression of genes known to relate to the desired endogenous physiological pathway). Alteration in the phenotype of interest can be detected directly (e.g., as in the case of a bioassay that detects changes in growth), indirectly (e.g., through a bioassay for alteration in the expression pattern of a reporter that correlates to that phenotype), or by assaying for an alteration in an expression profile of one or more genes (which may itself be the phenotype of interest, or alternatively may indirectly reflect the phenotype). In some embodiments of the invention, the results of the first phenotypic interaction are used to force the second round of protein interaction and phenotypic assays to converge upon a smaller, more focused group of phenotypic probes. In other embodiments, one may proceed with only one round of protein interaction and phenotypic assay when the candidate target was previously known.

The above-summarized detection and validation methodologies provide a parallel screening protocol for establishing the physiological relevance of putative therapeutic targets. First, testing with at least two “independent” probes—i.e., probes that are identified in separate assays, and which can be optionally derived from separate libraries—reduces or eliminates false positives that derive from artifactual interactions. Second, testing with at least two “phenotypic” probes (or at least one when the candidate target was previously known) substantially increases the likelihood that the binding partner is physiologically relevant, because interaction with probes that causes an alteration in the phenotype of interest provides strong validating evidence that the protein-ligand interaction is in fact linked to the endogenous cellular pathway(s) related to the phenotype. Because both the first and subsequent probes are independently shown to be physiological effectors of the same or related phenotypic trait, any endogenous cellular component that interacts with both probes is highly likely to be a true positive—i.e., to be involved in a physiologically relevant endogenous cellular pathway in the cell. When applied to a previously known candidate target, the methodology works in the same manner to establish whether the candidate target is physiologically relevant. Thus, when the phenotype of interest is selected so as to relate to, e.g., a disorder or disease of interest, the inventive methodology provides strong evidence that any endogenous cellular component thus validated is a bona fide therapeutic target. That validated target, in turn, has many uses, including (i) screening for small molecules that bind to the target and exert a therapeutic effect, (ii) elucidating physiological pathways, (iii) identifying gene(s) that encode or relate to the target, and (iv) providing the basis for diagnosing related physiological abnormalities.

In particular aspects of the claimed invention, the phenotypic probes will be “perturbagens.” The nature and use of perturbagens have been described in more detail in co-pending, co-owned applications U.S. Ser. No. 08/699,266, filed Aug. 19, 1996 (“Selection Systems For The Identification Of Genes Based On Functional Analysis”), W098/07886, and in U.S. Ser. No. 08/812,994, filed Mar. 4, 1997 (“Methods For Identifying Nucleic Acid Sequences Encoding Agents That Affect Cellular Phenotypes”), the disclosures of which each are specifically incorporated by reference in their entirety. Succinctly, perturbagens include proteinaceous molecules (proteins, protein fragments or domains, polypeptides or peptides) or nucleic acid moieties that act in a transdominant mode by interacting with endogenous components of a target cell (rather than on alleles of genes), and thereby interfering with normal cellular function. The perturbagens typically interact with proteins or polypeptides that reside in or on the therapeutic target cell, or with mRNA or DNA of the target cell. That therapeutic target cell is often a mammalian cell, in some embodiments, a human cell that relates to cancer, viral infection or metabolic disorders such as diabetes.

Certain aspects of the inventive methods feature the yeast two-hybrid assay system, although the claimed inventions are generally applicable to other methodologies for detecting protein-ligand interactions. In one exemplary preferred embodiment for detection and validation, at least two rounds of protein interaction assays and two independent sets of phenotypic probes are used to establish the physiological significance of any putative endogenous target molecule. In this particular preferred embodiment, one cycles between verifying the physiological significance of a perturbagen or other such probe, and identifying endogenous proteinaceous components that bind to those physiologically relevant probes. When the identity of the candidate target molecule is known in advance, then only one cycle of protein interaction assay and bioassay is required. Generally, the inventive methods are applicable to both single candidate targets and to pools of such candidate targets. The validated targets obtained thereby are optionally used to screen for therapeutic compounds that interact with those targets in a manner akin to the phenotypic probe(s), and more preferably in a high throughput screen for disruption of the target/probe pair.

Optionally, the “prey” or interaction target used in the first yeast two-hybrid assay may be used as the “bait” or interaction probe in a subsequent yeast two-hybrid assay step. The basic inventive method may also include an additional step of counter-selecting against interaction probes that self-activate. This additional step provides still further advantageous elimination of false positives that are assay artifacts.

With the present invention, it is possible to identify and/or validate protein interactions based on a phenotype, and test these interactions en masse in a high throughput manner to pinpoint the true, physiologically relevant interactions.

### BRIEF DESCRIPTION OF THE DRAWINGS

FIG. 1 is a pictorial flow chart summarizing the basic methodology of identifying physiologically validated target molecules with phenotypic probes.

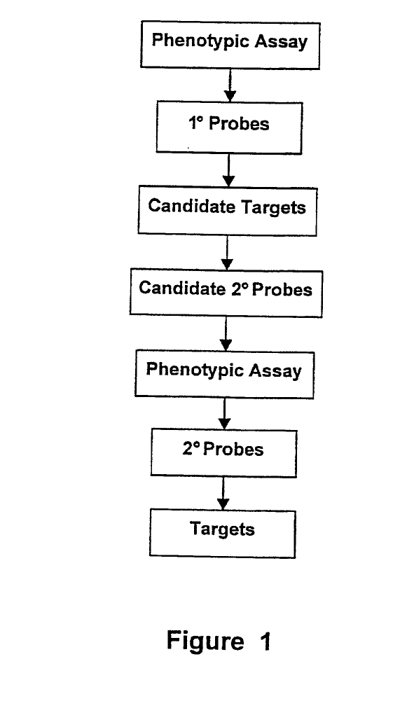

FIG. 2 is a pictorial flow chart summarizing a method of identifying physiologically validated target molecules utilizing phenotypic probes (identified with physiological assays) and yeast two-hybrid protein interaction assays.

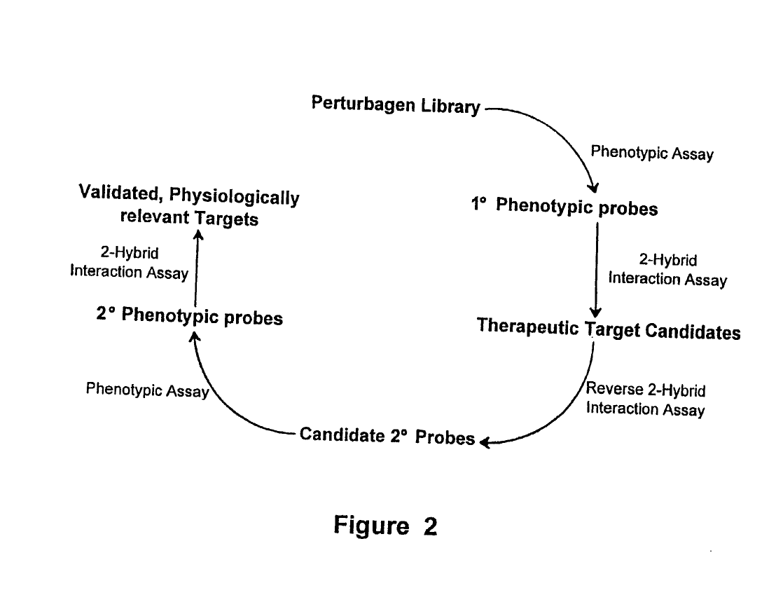

FIG. 3 is a diagram of representative yeast two-hybrid reporter constructs that are designed for use in a Gal4-based reporter system: (1) pVT85 (The shaded region represents the upstream activating sequence (UAS) and part of the 5′ untranslated region (UTR) of the yeast Gal2 gene spanning nucleotides 9-854 5′ of the Gal2 gene. The open box represents the first 9 nucleotides of the 5′ UTR, entire coding region and first 81 nucleotides of the 3′ UTR of the URA3 gene. Regions denoted with single lines represent chromosome 2 DNA flanking the reporter ending at nucleotide 473885 (5′ region) and starting at nucleotide 469705 (3′ region)); (2) pVT87 (schematics as for pVT85, except that the nucleotides 9-535 5′ of the Gal1 gene is used, and the open box represents the first 10 nucleotides of the 5′ UTR and entire coding region of the His3 gene. Regions denoted with single lines represent chromosome 15 DNA flanking the reporter ending at nucleotide 721943 (5′ region) and starting at nucleotide 722607 (3′ region)); (3) pVT88 (schematics as for pVT87, except that the nucleotides 38-242 5′ of the Gal7 gene is used, and the open box represents the first 10 nucleotides of the 5′ UTR and entire coding region of the His3 gene); and (4) pVT89 (the shaded region represents the UAS, 5′ UTR and a portion of the coding region of the Gal1 gene in total spanning nucleotides −535 to +87 of the Gal1 gene. The open box represents the coding region of the LacZ gene fused to the Lys2 3′ UTR).

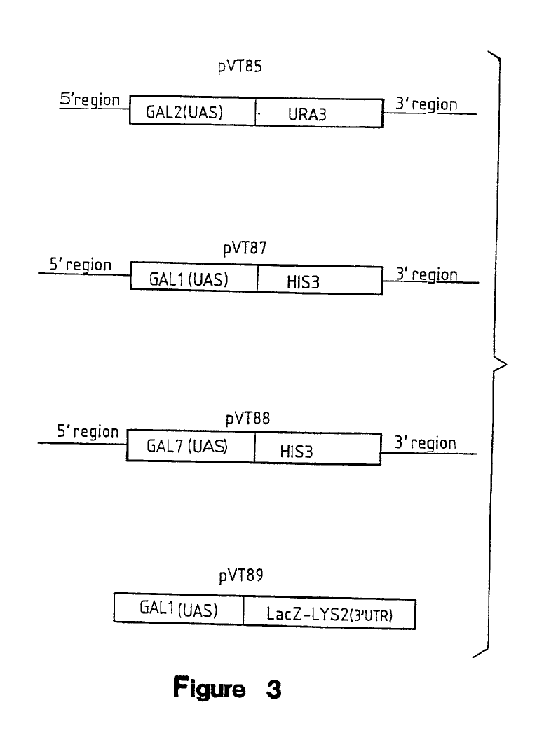

FIG. 4 is a diagram of representative yeast two-hybrid reporter constructs that are designed for use in a LexA-based reporter system: (1) pVT86 (the shaded region denotes eight LexA operators embedded within the Gal1 UAS; the open box represents the first 9 nucleotides of the 5′ UTR, entire coding region and first 81 nucleotides of the 3′ UTR of the Ura3 gene. Regions denoted with single lines represent chromosome 2 DNA flanking the reporter ending at nucleotide 473885 (5′ region) and starting at nucleotide 469705 (3′ region); and (2) pVT90 (schematics as in pVT86, but the open box represents the LacZ gene).

FIG. 5 is a diagrammatic representation of plasmid vector pVT562.

FIG. 6 is a diagrammatic representation of plasmid vector pVT592.

FIG. 7 is a diagrammatic representation of plasmid vector pVT560.

FIG. 8 is a diagrammatic representation of plasmid vector pVT725.

### DETAILED DESCRIPTION OF THE PREFERRED EMBODIMENTS

### Overview of the Screening Methodology

Developing new therapeutic agents and identifying genes that are involved in disease pathways share a common prerequisite—the need to delve into the molecular workings of the therapeutic target cell (e.g., a cancer cell) and to identify endogenous cellular components that are suitable targets for further research. These cellular components may be part of an endogenous intracellular pathway that is related to a particular cellular abnormality, disorder or disease, for example melanoma, breast cancer, diabetes or viral infection. Alternatively, the endogenous cellular components may be secreted proteins or cell-surface or membrane components, such as proteins, glycoproteins or phospholipids, that participate in cell-signaling, cell-recognition or other endogenous cellular pathways. Non-limiting examples of such target cellular components include: kinases, phosphatases, G proteins, G protein-coupled receptors, cyclins, transcription factors and integrins. Modification or disruption of such endogenous intracellular or cell-surface pathways (collectively referred to herein as “physiological pathways”) may lead to a cellular abnormality, disorder or disease state. Thus, identifying any endogenous cellular components that are involved in or related to such physiological pathways may provide valuable insight into diagnosis or treatment.

The first step in identifying and/or validating relevant endogenous cellular interactions is to select a host cell for use in an assay that is representative of a therapeutic target cell (e.g., HS294T/melanoma). When one seeks to identify new candidate targets, DNA encoding phenotypic probes (e.g., perturbagens) is preliminarily introduced into the assay cell line. The perturbagen expression products then specifically interact with one or more endogenous cellular components to affect or perturb (i.e., increase, decrease or otherwise alter) the normal activity of one or more of the endogenous components in the host cell. Altering the behavior of the endogenous components may, in turn, alter or perturb a physiologically relevant pathway associated with those components. That perturbation can be detected by a correlative change in a phenotypic characteristic (also referred to as the “phenotype of interest” or “phenotypic state”) of the host cell. A “phenotypic” characteristic refers to a measurable or monitorable indicia of the physiological state or appearance of the cell. The selected phenotypic characteristic may be used directly as a precise indicator (e.g., as a screening, or preferably, a selection criterion) of the physiological state of the targeted pathway. Alternatively, the phenotypic state of the host cell may be monitored through detecting changes in the level of expression of a separate reporter gene that is not necessarily part of the physiological pathway of interest but which, nonetheless, correlates to the phenotype. As another alternative, the phenotypic state may be or be reflected by changes in the expression profile of one or more gene in the host cell.

Next, if required to identify candidate targets for subsequent validation, endogenous cellular components that interact with the phenotypic probe are identified using standard biochemical or quasi-genetic protein interaction assay techniques (e.g., two-hybrid systems in yeast, bacteria or mammalian cells). This interaction assay completes the first round of phenotypic target screening (i.e. a first physiological assay in the host cell to identify a phenotypic probe, followed by a first protein-ligand interaction assay to identify endogenous cellular components that interact with that first phenotypic probe), and yields a first pool of interacting cellular components, termed herein “putative therapeutic target molecules” (also referred to herein as putative or candidate targets, therapeutic target candidates or the candidate target library). These putative targets may be entirely true positives (i.e. endogenous cellular components that relate to a physiological pathway of interest). Alternatively, and more likely, the set of components may contain a high percentage of false positives identified on the basis of an artifactual interaction in the protein interaction assay. Or, the set of components may contain false positives in that they are interactions that do occur in the host cell, but that do not have relevance to the endogenous physiological pathway of interest.

To segregate true positives from false positives (i.e., to identify physiologically relevant endogenous cellular interactions), an independent cycle of phenotypic evaluation is utilized. This step validates the physiological relevance of these putative therapeutic target molecules. Generally, in preferred embodiments, this technique involves using standard protein-ligand interaction assays to expose the above-described putative therapeutic target molecule(s) to an independent pool of putative confirmatory probes (in some embodiments also referred to herein as candidate secondary probes)—for example, an independent putative perturbagen library. (Note that in such embodiments, this set of probes has not yet been established as “phenotypic” probes, in that the ability to perturb the host cell in an appropriate physiological assay has not yet been evaluated.) From this independent protein-ligand interaction assay, those sequences encoding putative perturbagens probes (e.g., candidate secondary probes) that bind to the putative target molecules are isolated either by PCR or by plasmid isolation techniques, re-cloned into expression vectors suitable to drive expression in the host cells used in the phenotypic assay, introduced into those host cells, and subjected to another round of phenotypic assaying. In embodiments employing a second phenotypic assay, it may be identical to the assay used in the first round of phenotypic screening, or it may be chosen to monitor and select a closely related phenotype of interest. Such second rounds of phenotypic screening cull a second independent set of physiologically significant, confirmatory phenotypic probes from the sublibrary of putative confirmatory probes that bound to the candidate target molecule(s).

Finally, in this preferred embodiment, if a preliminary cycle of phenotypic evaluation was required to generate a pool of putative targets, or if a group of such putative targets were known in advance, then individual probe/target pairings may be identified by performing another independent protein interaction assay. To do so, the confirmatory phenotypic probes are exposed to the pool of putative therapeutic target molecules and, again using standard protein-ligand interaction techniques, members of the library of putative therapeutic target molecules that bind to particular confirmatory phenotypic probes are identified and isolated. In embodiments requiring two cycles to both identify and validate previously unknown candidate targets, binding with the candidate target molecules is used as a criterion for narrowing the pool of putative confirmatory probes that are subjected to the second phenotypic assay, and thus the second round of screening forces a convergence upon a more focused group of secondary phenotypic probes. Alternatively, if only one individual putative target is validated by the first round of phenotypic screening, the final protein-interaction step is not required to correlate that individual phenotypic probe to the corresponding endogenous cellular binding partners.

In other preferred embodiments, the above-described steps of (i) phenotypic screening and (ii) screening for protein-ligand interaction are reversed. Specifically, the second, independent pool of putative confirmatory probes (typically, a perturbagen library) is first phenotypically assayed to select a sublibrary of confirmatory phenotypic probes. Only then are the secondary probes exposed to the library of putative therapeutic target molecules. One may advantageously use either type of embodiment, based upon the relative speed and/or ease of the selected phenotypic assay protocol vs. the selected protein interaction assay protocol, and/or based on the number and binding characteristics of the perturbagens identified in the first and second rounds of phenotypic target screening.

### Phenotypic Assays

Discriminating between interactions that are relevant to the endogenous physiological pathway of interest and those that are irrelevant advantageously uses probes that have phenotypic relevance—i.e., the ability to directly or indirectly correlate a phenotypic change to a particular endogenous cellular interaction by perturbing the normal physiologic function of a host cell that has been selected to represent the ultimate therapeutic target cell.

When the phenotypic change is monitored directly, the action of the phenotypic probe or perturbagen at the molecular level within the host cell results in a readily measurable or identifiable change. Such changes can include, but are not limited to; (i) cell growth in the presence of various cytotoxic or cytostatic stimuli such as, e.g.,yeast mating pheromone, retinoic acid, chemotherapeutic agents like cisplatin and growth factor deprivation such as insulin withdrawal (ii) cell death or cell cycle arrest in the presence of specific stimuli or agents such as; e.g., tumor suppressors such as p16; (iii) behavioral changes such as gain or loss of adhesion; (iv) changes in gross cellular morphology, for example alterations that are visible microscopically, or (v) directly observable changes in protein expression, e.g. cell-surface proteins. Similarly, changes in gene expression profiles may constitute or reflect a phenotypic state. Such altered profiles may be detected in the host cell using, e.g., microarray technology familiar to those of ordinary skill in the art.

When the phenotypic change is monitored indirectly, the phenotypic state is monitored via an appropriate surrogate—e.g., a reporter gene that correlates to but may be independent of the phenotype. Examples include, without limitation, (i) induction, in the presence of defined stimuli, of specific reporter genes (e.g., fluorescent materials such as a GFP) fused to cis regulatory sequences whose expression is linked to a phenotype of interest such as apoptosis; and (ii) reduction in reporter gene expression in the presence of defined stimuli, again using a reporter whose expression is linked to the phenotype of interest. Such indirect monitoring is described in detail in U.S. Ser. No. 08/812,994, supra, incorporated herein by reference.

Representative assays that monitor exemplary phenotypic characteristics are listed in Table 1.

TABLE 1
Representative Phenotypic Assays

| | Cell line used in | Therapeutic |
|---|---|---|
| Phenotype | Assay | target cell type |
| Yeast mating | Yeast | Screening model for |
| pheromone response | | growth in human cells; |
| | | model antifungal |
| | | screening system |
| FUS1-up/down | Yeast | Screening model for |
| regulation | | growth in human cells; |
| | | model antifungal |
| | | screening system. |
| P16 tumor suppressor | HS294T (human | Metastatic melanoma; |
| resistance/release | Melanoma cell line), | also a generally |
| from apoptosis | and other carcinoma | applicable screening |
| | cell lines | model for other |
| | | carcinomas. |
| P16 tumor suppressor | WM35 (human | Late melanoma; also a |
| resistance/release | Melanoma cell line), | generally applicable |
| from apopsosis | and other carcinoma | screening model for other |
| | cell lines | carcinomas. |
| Serum/insulin | WM1552C (human | Early melanoma; also a |
| independent growth | Melanoma cell line), | model for other early- |
| | and other carcinoma | stage carcinogenic cell |
| | cell lines | lines that are growth- |
| | | factor independent. |
| Cis-Platin resistance/ | WM35 and other | Early melanoma; also a |
| selection based on | Carcinoma cell lines | generally applicable |
| cellular resistance to a | | model for chemotherapy- |
| model chemothera- | | resistant carcinomas. |
| peutic agent. | | |
| Foci development/loss | NIH3T3 (mouse | Model for any human |
| of contact Inhibition | Fibroblast cell line) | cancer cell for which loss |
| | | of contact adhesion is a |
| | | representative feature. |
| Adenovirus resistance | 293 (embryonic | Model for adenovirus |
| (growth inpresence of | kidney cell line) | infections associated |
| adenovirus) | | with, e.g., the common |
| | | cold. Also a model |
| | | system for other viruses |
| | | that infect a variety of |
| | | human cells. |
| Serum-independent | WM1552C and other | Early melanoma; also a |
| growth (i.e., cells | Carcinoma cell lines | model for other early- |
| capable of growing | | stage carcinogenic cell |
| without normally- | | lines that are growth- |
| required growth | | factor independent. |
| factors) | | |
| Retinoic acid | WM35 and other | Late melanoma; also a |
| resistance (causes | Carcinoma cell lines | general model for |
| cell death). | | treatment-resistant |
| | | carcinomas. |
| Retinoic acid | HS294T and other | Metastatic melanoma; |
| resistance (causes | Carcinoma cell lines | also a general model |
| cell death). | | for treatment-resistant |
| | | carcinomas. |
| P16 up-regulation | All the above, and | Generally applicable |
| (induction of P16 | other carcinoma cell | model for carcinomas. |
| tumor suppressor | lines | |
| expression) | | |
| Retinoic acid response | WM35 and other | Early melanoma; also a |
| element up-regulation | Carcinoma cell lines | general model for |
| | | treatment- |
| | | resistant carcinomas. |
| DyePKH26: a model | All the above, and | Generally applicable |
| dye that normally | other carcinoma cell | model for carcinomas. |
| partitions into the | lines | |
| membrane during cell | | |
| division; detects | | |
| changes in cell | | |
| division. | | |
| Caspase dyes | All the above, and | Generally applicable |
| (substrates for | other carcinoma cell | model for carcinomas. |
| proteases activated | lines | |
| during | apoptosis); | |
| detects apoptosis. | | |
| Binding of Apo2.7 | All the above, and | Generally applicable |
| (an antibody that | other carcinoma cell | model for carcinomas. |
| recognizes an epitope | lines | |
| present on dying | | |
| cells): detects cell | | |
| death, | | |
| “Floaters” (cells that | All the above, and | Generally applicable |
| detach from the plate | other carcinoma cell | model for carcinomas. |
| or other substrate): | lines | |
| detects cell death. | | |

In some instances, the cell line used in the phenotypic assay (also termed herein the “phenotypic assay host cell” or simply “host cell”) may actually be the identical to the therapeutic target cell (i.e., the phenotypic assay may be performed directly on the cancer cells of interest). In other instances, the phenotypic assay host cell may be of the same origin as the therapeutic target cell, but is modified in some way for laboratory use. In still other instances, the phenotypic assay host cell may be selected so as to be representative of some aspect of the therapeutic target cell, but is unrelated to that cell (e.g., using yeast cells to model human cells, or using embryonic kidney cell line 293 as a model for viral infection of mammalian cells in general). In any event, a wide variety of suitable phenotypic assays and host cells are known to those of skill in the art, and Table 1 is only a representative sampling of such cells and assays.

### Phenotypic Probes

An exogenous molecule that effects a change in the phenotype of interest in a selected host cell is termed a phenotypic probe. As explained in U.S. Ser. Nos. 08/699,266 and 08/812,994, supra, perturbagens are a class of molecules with great utility as phenotypic probes. As such, perturbagens interact with the endogenous physiological pathways of a cell and cause correlative phenotypic changes that are useful surrogates for tracking disruption of the endogenous pathways within the target cell. As described in more detail in the above-referenced, related applications and elsewhere herein, these phenotypic changes are detected using appropriate assays.

Perturbagens may be proteinaceous molecules (proteins, protein fragments or domains, polypeptides or peptides), nucleic acid moieties that interact with endogenous components of a target cell, or other organic or inorganic compounds. Proteinaceous perturbagens may be presented to the system of choice as products of expression libraries comprised of, e.g., synthetic DNA, cDNA or fragmented, sheared or digested genomic DNA (“perturbagen libraries”). Perturbagens may be expressed in cells without any additional sequences joined to them, or alternatively may be fused to other molecules. For example, one or more polypeptide sequences may be fused to the perturbagen to increase stability of the perturbagen in the assay system and/or to provide an easily detectable feature, such as fluorescence. Examples of such fusion moieties include GFP, LacZ or Gal4. Details are provided in co-pending, co-owned U.S. Ser. No. 08/965,477, “Methods And Compositions For Peptide Libraries Displayed On Light-Emitting Scaffolds,” the disclosure of which is incorporated herein in its entirety.

Once expressed within the target cell, perturbagens may induce a phenotypic state in the host cells that tracks or mimics a genetic mutant or epigenetic state. Any alteration in the target cell is detected through monitoring correlative changes in a reporter gene, or in another appropriate characteristic such as cellular growth or morphology, or expression of a marker. If a reporter gene is used, it is chosen to correlate with and thus reflect the relevant phenotypic state as closely as possible. The reporter is expressed in the host cells at a level sufficient to permit its rapid and quantitative determination.

### Disruption of Previously Unidentified Endogenous Pathways and Components

The methods of the invention preferably may advantageously be applied to identify components of previously uncharacterized biochemical or physiological pathways in the target cell, for example, genetic discoveries of pattern formation genes in Drosophila, C. elegans, and mammals. In other embodiments, the methods of the invention may advantageously be applied to isolate a previously unidentified endogenous component of an endogenous pathway that previously had been at least partially characterized. Examples include using revertants from p16-mediated cell cycle arrest to identify downstream components of the p16 pathway involved in growth control.

### Reporter Genes

Numerous reporter genes have been appropriated for use in expression monitoring, and are thus suitable for indirect monitoring of the phenotypic state of the cell. A reporter comprises any gene product for which screens or selections can be applied. Reporter genes used in the include the LacZ gene from E. coli (Shapiro S. K., Chou J. et al, Gene Nov.; 25: 71-82 (1983)), the CAT gene from bacteria (Thiel G., Petersohn D., and Schoch S., Gene Feb. 12; 168: 173-176 (1996)), the luciferase gene from firefly (Gould S. J. and Subramani S., 1988), the native GFP gene from jellyfish (Chalfie, M. and Prasher D. C., U.S. Pat. No. 5,491,804), modified or mutated forms of GFP (Abedi et al (1998)), GFP from other organisms (Prolume, Clontech), and DsRed (Clontech). This set has been primarily used to monitor expression of genes in the cytoplasm. A different family of genes has been used to monitor expression at the cell surface, e.g. the gene for lymphocyte antigen CD20. Normally a labeled antibody is used that binds to the cell surface marker (e.g., CD20) to quantify the level of reporter (Koh, J., Enders, G. H. et al., 1995).

Native GFP is a member of a family of naturally occurring fluorescent proteins, whose fluorescence is primarily in the green region of the spectrum. Native A. Victoria GFP has been developed extensively for use as a reporter and several modified or mutant forms of the protein have been characterized that have altered spectral properties (e.g., Cormack, B. P., Valdivia R. H. and Falkow, S., Gene 173: 33-38 (1996)). (Both native A. Victoria GFP and such related molecules are encompassed within the term “GFP” as used herein) High levels of GFP expression have been obtained in cells ranging from yeast to human cells. GFPs are robust, all-purpose reporters, whose expression in the cytoplasm can be measured quantitatively using a flow sorter instrument such as FACS.

Of these reporters, autofluorescent proteins (e.g., GFP) and the cell surface reporters are potentially of greatest use in monitoring living cells, because they act as “vital dyes.” Their expression can be evaluated in living cells, and the cells can be recovered intact for subsequent analysis. Vital dyes, however, are not specifically required by the methods of the present invention. It is also very useful to employ reporters whose expression can be quantified rapidly and with high sensitivity. Thus, fluorescent reporters (or reporters that can be labeled directly or indirectly with a fluorophore) are especially preferred. This trait permits high throughput screening on a flow sorter machine such as a fluorescence activated cell sorter (FACS).

The selected reporter gene may also advantageously act as a scaffold for a desired sequence (e.g., DNA encoding a perturbagen). GFP, for example, can be used as such a scaffold, and the structure of the expressed polypeptide serves as a stabilizing polypeptide for the perturbagen insert. The perturbagen sequence can either be inserted at or near the N- or C-terminus of the GFP scaffold, or alternatively can be inserted into a suitable internal site. The use of GFP as a stabilizing polypeptide scaffold is described in U.S. Ser. No. 08/965,477, supra, and is incorporated by reference herein.

### High-throughput Protocols

Preferably, individual cell phenotype states are determined via a selection device or method that permits rapid, quantitative measurement of the expression levels of the reporter, selection molecule or other selection criterion on a cell-by-cell basis. As used herein, the phrase “high throughput” refers to cell sorts of at least 1×10³ cells per hour, and more preferably 1×10⁷ cells per hour. High throughput screens, selections or assays generally involve techniques that permit numerous cells or reactions to be analyzed either in parallel, or in a rapid serial fashion. For example, the flow sorter is a high throughput serial device since it can examine roughly 1×10⁸ cells per hour. Parallel analyses may utilize automated or semi-automated formats, and high throughput applications achieve complete preparation and assay of approximately 10,000 samples or more per 10 days' continual operation.

In one preferred high-throughput embodiment to identify and validate targets, cells with a desired phenotypic profile are isolated, for example, by flow cytometry or other appropriate separation technique. Cell separation may be performed on the basis of any suitable selection criteria, such as fluorescence or magnetic characteristics. The resident probes that correlate to that phenotype are recovered, for example by PCR amplification of the resident DNA sequences that encode them. These recovered probes, in turn, are used to isolate their endogenous binding partners. Generally, this can be accomplished using standard protein interaction techniques—for example, by biochemical binding assays or yeast two-hybrid assays. DNA encoding a pool of putative therapeutic target molecules that are perturbagen binding partners is then isolated, for example by standard techniques of DNA recovery followed by PCR amplification using flanking sequences as primer-binding sites, or by plasmid isolation techniques familiar to those skilled in the art.

### Embodiments Using Yeast Two-hybrid Technology

The phenotypic evaluation steps of the present invention require some method for detecting interactions between proteinaceous perturbagen probes and endogenous target molecules. The yeast two-hybrid technique is one such method. When the yeast two-hybrid technology is applied in the context of the present invention, it may be used to detect interaction between putative or actual phenotypic probes and putative or actual endogenous therapeutic targets. The general strategy and experimental details of this method are familiar to those of ordinary skill in the art.

In one embodiment for identifying and validating endogenous cellular targets, a population of putative endogenous therapeutic target molecules is identified with a first set of phenotypic probes (e.g., perturbagens) by first introducing the initial library of putative perturbagen-encoding sequences into the cell line used in the phenotypic assay (e.g., human cell lines HS294T, WM35, or WM1552C—representative of human melanoma therapeutic target cells). The cells containing the perturbagen DNA are then subjected to a phenotypic selection or assay, such as a protocol for selecting variant cells that grow in the presence of a stimulus that kills the vast majority. Cells having the desired phenotype are identified and segregated via standard techniques such as FACS or growth-based characteristics. From these phenotypically culled cells, a primary set of phenotypic probes that altered the physiological state of the cells are recovered. The phenotypic probe is presumed to have exerted its phenotypic effect through an interaction with a relevant endogenous component of the target cell.

Next, a first yeast two-hybrid protein interaction assay is performed to identify a pool of endogenous cellular components from, e.g., the phenotypically culled cells that interact with the first set of phenotypic probes. These cellular components are thus identified as putative therapeutic target molecules.

As one non-limiting example, each of the physiologically relevant primary perturbagens, described above, is cloned as a fusion with the DNA binding domain of a transcription factor, e.g., Gal4, as the bait or interaction probe for a two-hybrid search. These phenotypic bait constructs are introduced into an appropriate yeast strain, for example any strain suitable for co-transformation, or having the ability to mate to yeast cells of the opposite mating type and capable of serving as a vehicle for propagation of and selection for the transcription factor (e.g., Gal4) fusion bait-encoding plasmid. Next, this strain is mated to a second strain of yeast that harbors the prey library that has been cloned in frame with an appropriate AD. As one non-limiting example, that library is constructed so as to contain all possible protein domains present in the target cell or organism of interest. This can be accomplished (in whole or in large part) by the use of fragmented gDNA or random-primed cDNA cloned into the appropriate yeast two-hybrid expression vector.

The yeast cells containing the prey constructs are then mated to yeast cells that harbor the perturbagen-containing bait constructs. The resultant mated cells are plated on an appropriate medium (e.g., a medium designed to detect the particular marker activity that is associated with the AD/BD complex). Yeast cells expressing the marker gene are recovered. An unspecifiable portion of these cells will evidence marker gene expression due to interaction between the bait (perturbagen) and the prey (endogenous cellular target candidates). These specific prey sequences comprise a sub-library of candidate perturbagen binding partners or targets. The candidate target sub-library can be recovered as pure DNA (absent the yeast) by PCR amplification using flanking sequences as primer-binding sites, or by plasmid isolation techniques familiar to those skilled in the art. Alternatively, the bait and prey constructs can be introduced into the same yeast cell and the resultant co-transformed yeast cells are plated on medium with recovery of cells expressing the marker gene.

These candidate target sequences may be amplified by reintroduction of the plasmids into E. coli, or by PCR. They may then be re-cloned as bait sequences (e.g., using the original GAL4 BD domain as a fusion partner) in preparation for a second round of yeast two-hybrid screening. Alternatively, the initial yeast two-hybrid screen can be performed in a “backwards” context in which the bait is linked to the AD domain and the prey (candidate target library) is linked to the BD domain. This obviates the need for a subsequent switch involving a re-cloning step to shuttle the prey into the bait vector. Instead, the initial candidate target sequences are recovered in a BD vector, and these can be used directly to screen a second library in an AD vector.

Optionally, self-activating sequences may be depleted from the second BD-fusion library prior to screening that library with the selected AD-fusion candidate targets. This can be accomplished using a negative selection, e.g., a selection against a URA+phenotype. The purpose of the negative selection is to remove from the second library sequences that self-activate; i.e., that can confer a URA+reporter phenotype in the absence of a second interacting protein that brings in the AD fusion. The sub-library, now depleted of self-activating sequences, can then be used as the prey in a second screen using the candidate targets as bait in order to identify secondary binders that are candidate perturbagens.

Regardless of the precise composition of the prey and bait constructs, a second protein interaction assay proceeds by mating appropriate yeast host cells in order to expose the bait and prey constructs. If, for example, the set of targets has been reconstituted as fusions to the BD moiety, they are mated en masse to a second prey library which may, e.g., contain perturbagen peptide sequences fused to the Gal4 activation domain. Once again, the transformed yeast are plated onto selective medium appropriate for the marker gene responsive to the BD/AD interaction construct, and pairs of target/prey interactors are recovered.

From this pool of second binding partners, a set of putative confirmatory probes (also referred to herein as candidate secondary probes) is recovered. These probes are PCR-amplified and cloned into a mammalian expression vector, for example a CMV-derived vector. The probes are then introduced into suitable host cells, for example those used in the original physiological assay, and subjected to a physiologic selection or screen in order to select a second pool of phenotypic probes. Those secondary phenotypic probe sequences that confer the same or similar physiological effect on the host cells as the original perturbagens (i.e., generate the phenotype of interest) are recovered. Finally, these secondary phenotypic probes are used to validate the physiological significance of members of the candidate target library. Candidate targets that bind to both the primary and secondary phenotypic probes are true targets. Because these endogenous targets are now shown to interact with two separate, independent sets of phenotypic probes, the target is an overwhelming choice for an in vivo therapeutic target.

The logical basis for matching a particular perturbagen to a particular target protein involves the identification of two independent effectors (e.g., perturbagens) that confer identical or similar physiological changes on host cells, and recognize the same target protein in protein-ligand interaction assays such as the yeast two-hybrid system. Using the series of steps described herein, it is possible to find two perturbagens that bind the same target protein, because the protein-ligand interaction steps force the perturbagens to converge on the same set of candidate targets (i.e., the second confirmatory effector perturbagen is isolated based on its ability to bind to a binding partner of the first perturbagen). In addition, the second confirmatory perturbagen (as well as the first) are identified by their physiological effect on cells. Thus, it becomes exceedingly unlikely that the common target of the two perturbagens is not the physiologically relevant target.

For previously identified candidate targets, the validation procedure may proceed directly with the second cycle of yeast two-hybrid assay and phenotypic assay, as described above.

### Yeast Two-hybrid Reporter Constructs

The yeast two-hybrid reporter gene is typically fused to the upstream promoter region that is recognized by the BD, and is selected to provide a marker that facilitates screening. Examples include the lacZ gene fused to the Gal1 promoter region and the His3 yeast gene fused to Gal1 promoter sequences. A variety of yeast two-hybrid reporter constructs are suitable for use in the validation methods of the present invention. Desirable criteria for these reporter constructs are that they provide a rigorous selection (i.e., yeast cells die in the absence of a protein-ligand interaction between the bait and prey sequences), or a convenient screen (e.g., the cells turn color when they harbor bait and prey sequences that interact). Examples include (1) the Ura3 gene, which confers growth in the absence of uracil and death in the presence of 5-fluoroorotic acid (5-FOA); (2) the His3 gene, which permits growth in the absence of histidine; (3) the LacZ gene, which is monitored by a calorimetric assay in the presence/absence of beta-galactosidase substrates; (4) the Leu2 gene, which confers growth in the absence of leucine; and (5) the Lys2 gene confers growth in the absence of lysine or, in the alternative, death in the presence of α-aminoadipic acid. These reporter genes may be placed under the transcriptional control of any one of a number of suitable cis-regulatory elements, including for example the Gal2 promoter, the Gal1 promoter, the Gal7 promoter, or the LexA operator sequences.

### Yeast Two-hybrid Host Strains

A variety of yeast host strains known in the art are suitable for use in the validation methods of the present invention. Desirable criteria for these host strains are that they can be mated to cells of opposite mating type (i.e., they are haploid), and they contain chromosomally integrated reporter constructs that can be used for selections or screens (e.g., His3 and LacZ). Generally, either Gal4 strains or LexA strains may be used with the appropriate reporter constructs. Examples include strains yVT96, yVT97, yVT98 and yVT99, described herein. Additionally, those of ordinary skill will appreciate that the host strains used in the present invention may be modified in other ways known to the art in order to optimize assay performance. For example, it may be desirable to modify the strains so that they contain alternative or additional reporter genes that respond to two-hybrid interactions.

### Embodiments Using Biochemical Binding Assays to Detect Interactions

As an alternative to using quasi—genetic methods such as the yeast two-hybrid methodology for detecting protein-ligand interactions, biochemical methods may be used to detect targets and to identify the second candidate perturbagens. For example, affinity purification techniques are well known to those of skill in the art. Proteinaceous probes such as perturbagens may be used as one component of an affinity purification, specifically to select perturbagen binding partners from a cellular extract. The perturbagens and associated endogenous cellular binding partners are isolated and collected for analysis by standard analytical methods. As one non-limiting example, mass spectrometric methods may be used to separate and characterize the endogenous perturbagen-binding proteins. By reference to sequence databases, the identity of the binding partner can be identified. This in turn facilitates isolation of cDNA encoding the binding partner for expression of suitable amounts of purified protein for use in a standard phage display procedure (e.g., expressed on phage). The purified candidate targets are exposed to a second set of candidate confirmatory probes. Probes from the phage display library that bind to the purified protein are recovered and subjected to an appropriate physiological assay, as described above. Finally, phenotypically relevant confirmatory probes are recovered as above. Candidate endogenous cellular targets that bind to these probes are identified and isolated, as above.

### Advantages of the Validation Methodology

The parallel phenotypic validation strategy of the present invention is a flexible and efficacious solution to the problem of false positives in protein interaction screening. Moreover, the invention provides a powerful tool for screening potential proteinaceous and non-proteinaceous therapeutic agents for their ability to effect a desired change in a physiologically relevant pathway.

One important feature of the invention described herein is that a particular putative therapeutic target molecule, known from protein-ligand interaction assays to interact with a perturbagen probe, can be linked with a high degree of certainty to a defined physiological pathway. Thus, it is possible to relate protein-ligand interactions to physiological pathways in cells, a link that is very difficult and time-consuming to establish normally. Without the approach described herein, each candidate target must be tested independently and painstakingly for a physiological role. This requires, for example, the production of antibodies or antisense constructs, their introduction into cells, and the monitoring of specific phenotypes.

The protocols of this invention are very advantageous because they permit high-throughput screening for endogenous targets of specific peptide or protein effectors that alter cellular physiology in defined ways. The specific advantages are twofold: first, the screening can be carried out en masse, obviating the need to painstakingly examine each candidate target individually. Second, false positives (e.g., spurious protein-ligand interactions identified via protein interaction assays) can be readily reduced or even eliminated. These advantages have important consequences. They sidestep a major obstacle in the upstream portion of the drug development process; namely, the difficulty of identifying validated, true targets of effector molecules (e.g., perturbagens). This is accomplished by tying specific perturbagen binding partners to physiological roles in the cell; that is, linking specific cellular proteins to definite biochemical/physiological pathways in cells.

It should be borne in mind that one of the major shortcomings associated with genomics and proteomics methods at present is the extreme difficulty associated with matching particular genes or proteins with physiological roles in cells. The methods described here provide a significant contribution to the solution to this problem. Using this technology, protein-ligand interactions can be assigned to specific physiologically relevant (and hence, medically relevant) pathways, and not merely catalogued. Once the physiological relevance of such protein-ligand interactions are established, such proteins (or their ligands) can readily be incorporated into known high throughput screening protocols, for use as reagents in identifying small organic molecules of potential therapeutic value.

The methods described herein thus provide a substantial advantage over the methodologies previously known to the art. Because any putative target candidate is linked to an endogenous cellular/physiological pathway of interest, which in turn is associated with a particular cellular abnormality, disorder or disease, its therapeutic utility is validated. This validation step provides additional efficiencies by reducing the size of the ultimate pool of targets that are to be subjected to additional research, and provides proven reagents for high throughput screening of, e.g., combinatorial chemistry libraries.

### EXAMPLE 1

### Creation and Characterization of the Phenotypic Probe Libraries and Candidate Target Libraries

(1) Construction of Perturbagen Libraries for Phenotypic Assays

Phenotypic assays may often utilize libraries of putative perturbagens which are constructed so as to provide the desired variety of genetic material for screening, in a vector that is suitable for the target cell used in the phenotypic assay. For example, when the therapeutic target cell of interest is a mammalian cell, or even more particularly a human cancer cell, the library must be constructed in a manner that allows for (1) introduction of the perturbagen library into the mammalian cell and (2) subsequent expression of the library in the mammalian target cell.

As one non-limiting example, a cDNA library that encodes potential perturbagens may be prepared according to the following procedure, using methods that are well known in the art. Double-stranded DNA is prepared from random primed MRNA isolated from a particular cell type or tissue, for example human placental tissue. Alternatively, randomly sheared genomic DNA fragments may be utilized. In either case, the fragments are treated with enzymes to repair the ends and are ligated into a suitable retroviral or episomal expression vector suitable for expression in, e.g., mammalian cells. One exemplary vector is pVT334 (described in WO 99/24617 [PCT/US98/23778, filed Nov. 5, 1998 as the PCT counterpart of priority document U.S. Ser. No. 08/965,477], the disclosures of which incorporated herein by reference), a retroviral vector that permits the expression of library clones as EGFP fusions from the CMV promoter. Such vectors can be packaged by standard procedures into infectious particles to facilitate introduction into human cells. The perturbagen-containing vectors are then introduced into E. coli and clones are selected. A number of individual clones sufficient to achieve reasonable coverage of the mRNA population (e.g., one million clones) is collected, and grown in mass culture for isolation of the resident vectors and their inserts. This process allows large quantities of the library DNA to be obtained in preparation for subsequent phenotypic screening and protein interaction assays, as described infra.

As a second general source of putative perturbagens, a synthetic DNA library encoding peptides of varying sizes can be prepared. For example, libraries encoding synthetic 15 amino acid (aa) peptides were created using the general method described in Abedi et al., Nucleic Acids Res. 26(2):623-630 (1998), and as described in co-pending U.S. patent application Ser. No. 08/965,477, supra, incorporated herein by reference. Briefly, DNA encoding randomly generated 15 amino acid peptides was synthesized and inserted into the XhoI and BamHI sites of a selected EGFP construct. These steps thus can create random peptide display libraries. Alternatively, targeted or engineered synthetic DNA libraries encoding “smart” perturbagens can be constructed. For example, a variety of DNAs encoding engineered variants of a known or suspected perturbagen may be readily constructed.

(2) Construction of Target Cell-specific Genetic Libraries

The protein interaction portion of the target validation methodology described herein requires presentation of a phenotypic probe to a library of proteins. In some embodiments, the proteins of interest may be particular to a selected target cell. In such cases, it is desirable to create and test a collection of endogenous cellular proteins derived from a cell line that is representative of the therapeutic target cell—e.g., HS294T, WM35 or WM1552C (melanoma). These endogenous proteins may be readily obtained by expressing a genetic library that is derived from the selected cell line. As one non-limiting example, the mRNA of the therapeutic target cell line is used to construct the therapeutic target library. Alternatively, cDNA libraries derived from fetal brain, liver or kidney may be prepared. The details of library construction, manipulation, and maintenance are as described above for the construction of a perturbagen cDNA library.

### EXAMPLE 2

### Creation and Characterization of Exemplary Yeast Two-hybrid Assay Components

Preparation of various yeast two-hybrid assay components—e.g., bait constructs, prey constructs, and host cells—are familiar to the art. The following are exemplary, non-limiting examples of such components.

(1) Suitable Yeast Vectors

Once the phenotypic probe (perturbagen) and target libraries are selected, each is incorporated into an expression vector that is appropriate for use in yeast. The target and perturbagen libraries are deployed as bait/prey libraries in appropriate bait and prey fusion constructs.

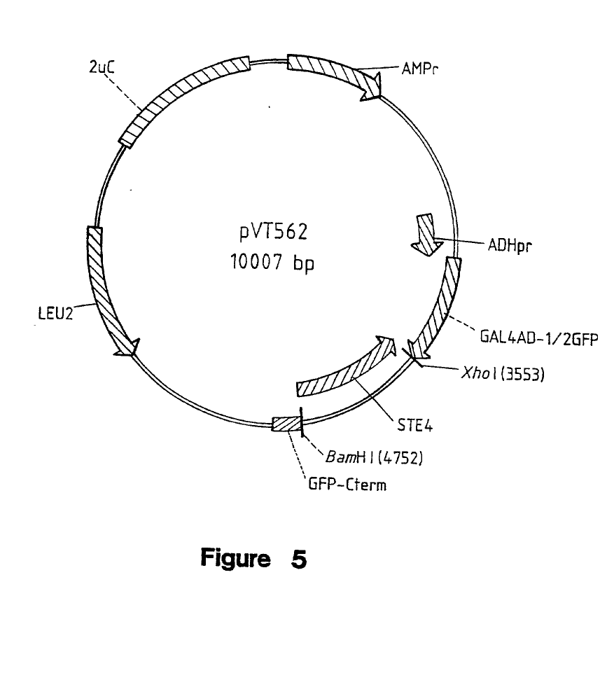

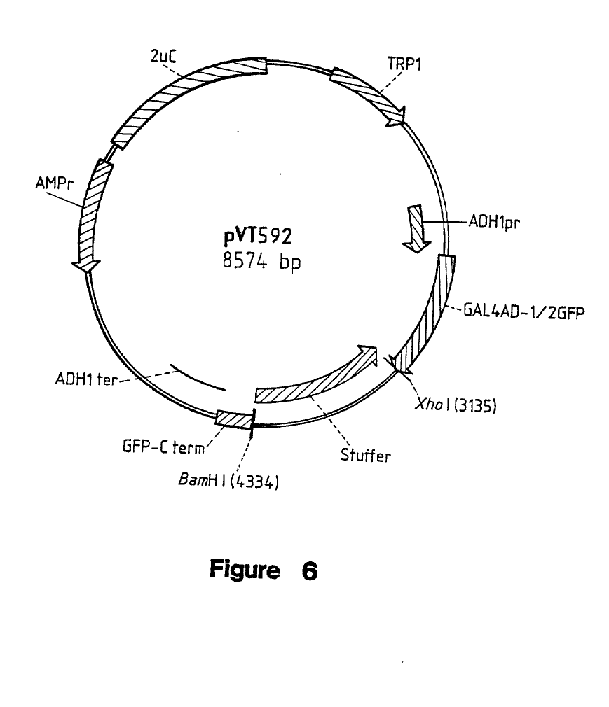

Suitable activation domain vectors for cDNA or gDNA-derived perturbagens include, e.g., pACT2. Suitable activation domain vectors for peptide perturbagens or peptide prey libraries include pVT562 (FIG. 5) and pVT592 (FIG. 6), which have a GFP scaffold protein with internal BamHI and XhoI sites for subsequent cloning of either the perturbagen or target sequences. The pVT562 and pVT592-based libraries are transformed into appropriate yeast strains, for example yVT97 and yVT98, respectively.

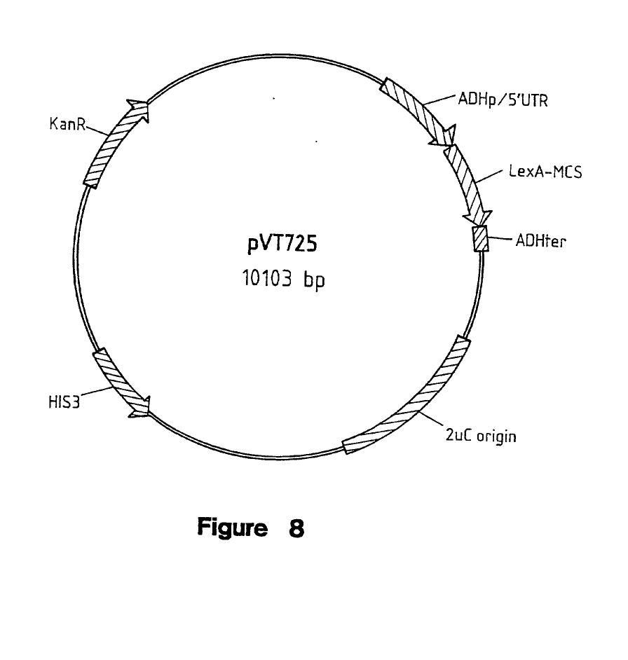

One exemplary binding domain vector is pVT725 which includes the bacterial LexA DNA binding protein fused to a multiple cloning site. This vector also contains the yeast His3 gene and the kanamycin resistance gene (KanR) for selection in yeast and bacteria, respectively. (FIG. 8).

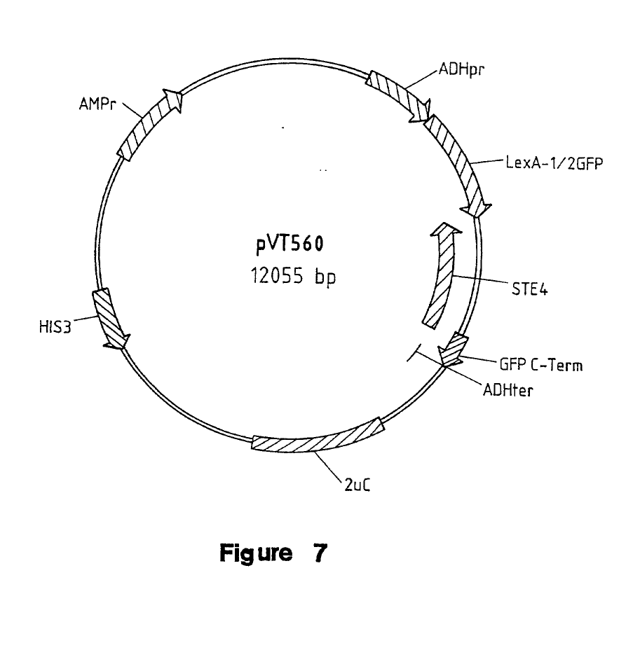

One exemplary vector for peptide prey libraries expressed as BD fusions is pVT560 (FIG. 7), which has a GFP scaffold protein with internal BamHI and XhoI sites for subsequent cloning of nucleic acid encoding, e.g., a library of peptide sequences. The pVT560-based libraries are transformed into appropriate yeast strains, for example yVT99 or yVT100.

Optionally, a prey library may be subjected to an additional step to eliminate self-activating sequences; e.g., yeast expressing peptides which self-activate transcription are removed via selection in the presence of 5-FOA creating a sub-library of yeast expressing non-activating sequences.

Identification of peptides capable of binding to perturbagen-target candidates is accomplished by mass-mating the peptide library expressing yeast with target-protein expressing yeast and selecting for growth on plates lacking histidine, leucine or uracil (depending on the selected reporter).

(2) Construction of Perturbagen Libraries for Yeast Two-hybrid Assays

As described in the previous Example, perturbagen libraries may be derived from a number of sources, including without limitation synthetic DNA inserts, gDNA or cDNA, and may be inserted into a scaffold protein, for example native or modified GFP. In order to screen the perturbagen library in a yeast two-hybrid assay, it must be incorporated into a suitable vector.

The vectors pVT560, pVT592 and pVT562 were constructed as follows.

Plasmid vector pVT560 (FIG. 7) was constructed by filling the BamHI and XhoI sites in pLexA (Clontech 98/99 p. 89) in separate steps using Klenow fragment. EcoRI was used to clone a GFP gene containing internal XhoI and BamHI restriction sites (as in pVT27, described in U.S. Ser. No. 08/965,477, supra, incorporated herein by reference) into the modified pLexA. The reading frame of GFP was such that it was in frame with the DNA binding domain in pLexA. Finally, a 1.2 Kb BamHI-XhoI stuffer fragment (containing 1194 coding bases of the yeast STE4 gene) was cloned into the GFP gene to yield pVT560.

Plasmid pVT592 (FIG. 6) was constructed by first generating a 1.5 kb fragment containing the ADH1 promoter, the Gal4 AD fused to a multiple cloning site, and the ADH1 3′ terminator by PCR from pACT2. Following PCR, overhanging ends were filled with Klenow fragment and the fragment was ligated into pRS124 (Sikorski & Hieter (1989)) that had previously been digested with PvuII and dephosphorylated with calf intestinal phosphatase (CIP). The resulting plasmid was digested with EcoRI, treated with CIP and ligated to an EcoRI fragment from pVT562 that contained the GFP gene (as well as a 1.2 kb XhoI/BamHI stuffer) such that GFP was in frame with the Gal4 AD, creating pVT592.

Plasmid pVT562 (FIG. 5) was constructed beginning with pACT2 (Clontech 97/98, p. 56) as follows. The BamHI and XhoI sites in pACT2 were filled in separate steps using Klenow fragment. EcoRI was used to clone a GFP gene containing internal XhoI and BamHI restriction sites (as in pVT27, supra) into the modified pACT2. The reading frame of GFP was such that it was in frame with the DNA binding domain in pACT2. Finally, a 1.2 Kb BamHI-XhoI stuffer fragment (containing 1194 coding bases of the yeast STE4 gene) was cloned into the GFP gene to yield pVT562. Perturbagen libraries are then cloned into the internal XhoI/BamHI site (or other desired internal site, as described in U.S. Ser. No. 08/965,477, supra, incorporated herein by reference). Alternatively, the perturbagen library may be cloned into positions at or near the N-terminus or C-terminus of a selected GFP. In these constructs GFP is expressed as a fusion protein with the perturbagens.

(3) Target Libraries for Yeast Two-hybrid Assays

As described in the previous Example, genetic libraries that are particular to the therapeutic target cell of interest may be constructed. Such a target library is incorporated into a vector that is suitable for use in yeast. One such exemplary vector is pACT2, which has a selectable TRP1 marker. The vector has an ADH promoter upstream of the target cell insert to drive its expression in a constitutive manner. Alternatively, commercial libraries may be utilized. Libraries suitable for performing two hybrid selections for the purpose of identifying candidate perturbagen targets can be obtained from several sources. For example, libraries for both LexA-based and Gal4-based two hybrid selections are commercially available from a variety of companies (e.g. Clontech and Origene).

(4) Reporter Constructs for Detecting Protein-ligand Interactions

Validating endogenous targets as physiologically relevant candidates for therapeutic intervention involves, inter alia, the creation and characterization of reporters for detecting protein-ligand interactions. The following are exemplary, non-limiting examples of such reporter constructs.

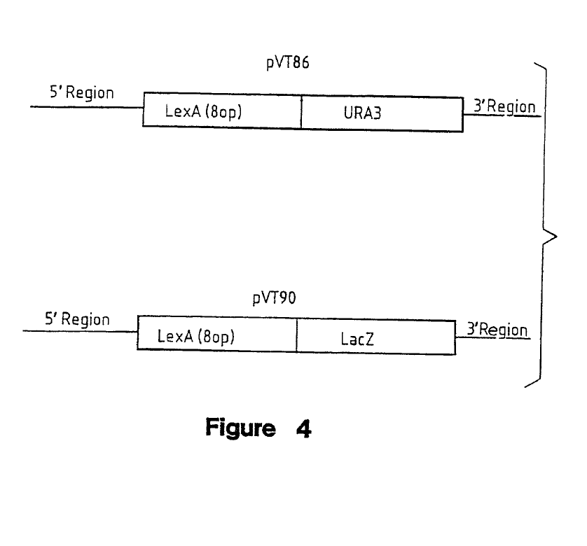

Reporter 1-(pVT85): This reporter comprises the URA3 gene under the transcriptional control of the yeast Gal2 upstream activating sequence (UAS). In order to facilitate integration of this reporter into the yeast chromosome in place of the Lys2 coding region, the Gal2-Ura3 construct is flanked on the 5′ side by the 500 base pairs that lie immediately upstream of the coding region of the LYS2 gene and on the 3′ side by the 500 base pairs that lie immediately 3′ of the coding region of the LYS2 gene. FIG. 4. The entire vector is also cloned into the yeast centromere containing vector pRS413 (Sikorski, R S and Hieter, P., Genetics 122(1):19-27 (1989) and can therefore be used episomally. This reporter is intended for use with a Gal4-based two-hybrid system, e.g., Fields, S. and Song, O., Nature 340:245-246 (1989).

Reporter 2-(pVT86): This reporter is identical to reporter #1 except that the GAL2 UAS sequences have been replaced with regulatory promoter sequences that contain eight LexA operator sequences (Ebina et al., 1983). FIG. 5. The number of LexA operator sequences in this reporter may either be increased or decreased in order to obtain the optimal level of transcriptional regulation. This reporter is intended to be used within the general confines of the LexA-based interaction trap devised by Brent and Ptashne.

Reporter 3-(pVT87): This reporter is comprised of the yeast His3 gene under the transcriptional control of the yeast Gal1 upstream activating sequence (UAS). In order to facilitate integration of this reporter into the yeast chromosome in place of the His3 coding region the Gal1-His3 construct is flanked on the 5′ side by the 500 base pairs (bp) immediately upstream of the His3 coding region and on the 3′ side by the 500 bp immediately 3′ of the His3 coding region. FIG. 6. The entire reporter is also cloned into the yeast centromere containing vector pRS415 and can therefore be used episomally. This reporter is intended for use with a Gal4-based two-hybrid system.

Reporter 4-(pVT88): This reporter is identical to Reporter 3 except that the His3 gene is under the transcriptional control of Gal7 UAS sequences rather than the Gal1 UAS. FIG. 7. The reporter is used with a Gal4-based two-hybrid system.

Reporter 5-(pVT89): This reporter contains the bacterial LacZ gene under the transcriptional control of the Gal1 UAS. The entire reporter will be cloned into a yeast centromere-using vector, e.g., pRS413, and is used episomally. FIG. 8.

Reporter 6-(pVT90): This reporter consists of the LacZ gene under the transcriptional control of eight LexA operator sequences. FIG. 9. As for Reporter 2, the number of LexA operator sequences in this reporter may either be increased or decreased in order to obtain optimal levels of transcriptional regulation. Two features of this reporter facilitate integration of the reporter into the yeast chromosome in place of the Lys2 coding region. First, it is flanked on the 5′ side by the 500 base pairs that lie immediately upstream of the coding region of the Lys2 gene and on the 3′ side by the 500 base pairs that lie immediately 3′ of the coding region of the Lys2 gene. Second, the neomycin (NEO) resistance gene has been inserted between the 5′ Lys2 sequences and the LexA promoter sequences. This reporter is used in conjunction with a LexA-based interaction trap, e.g., Golemis, E. A., et al., (1996), “Interaction trap/two hybrid system to identify interacting proteins.” Current Protocols in Molecular Biology, Ausebel et al., eds., New York, John Wiley & Sons, Chap. 20.1.1-20.1.28.

(5) Characterization of Reporter Constructs

Following construction, all reporters are characterized in appropriate yeast strains (described herein), utilizing centromere-based vectors. Specific parameters tested are as follows.

Reporter 1: Reporter 1 is characterized by the following steps: (a) detecting absence of growth on defined media lacking uracil and growth in the presence of 5-fluoroorotic acid (5-FOA); and (b) detecting growth in the absence of uracil and 5-FOA sensitivity in the presence of weak Gal4-transcriptional activators.

If desired, fine-tuning of this reporter in order to generate desired characteristics is accomplished by PCR-based mutagenesis of Gal2 UAS sequences combined with positive and negative selections involving uracil prototrophy and 5-FOA resistance.

Reporter 2: Reporter 2, comprised of the URA3 gene under the transcriptional regulation of 8 LexA operator (8op) was integrated in place of the LYS2 gene in the genome of strain EGY48, and integration was confirmed by the use the polymerase chain reaction (PCR). Following integration, the reporter was determined to have the following properties; (1) the reporter conferred a URA+phenotype to the host yeast strain in the presence of both strong and weak, LexA-fused, transcriptional activators; (2) the reporter conferred a URA+phenotype to the host yeast strain in the presence of a pair of interacting proteins, one expressed as a LexA fusion (p53) and the second fused to the B42 activation domain (Large T-antigen); (3) the reporter did not display a URA+phenotype in the presence of LexA fusions that do not normally activate transcription; (4) the reporter conferred a 5-FOA−phenotype to the host yeast strain in the presence of both strong and weak, LexA-fused, transcriptional activators; (5) the reporter conferred a 5-FOA−phenotype to the host yeast strain in the presence of a pair of interacting proteins, one expressed as a LexA fusion (p53) and the second fused to the B42 activation domain (Large T-antigen); (6) the reporter displayed a 5-FOA+phenotype in the presence of LexA fusions that do not normally activate transcription; and (7) the reporter was used successfully to cull self-activating sequences from a pVT560-based peptide library by selecting for those library clones able to grow in the presence of 5-FOA.

Reporters 3 and 4: Reporters 3 and 4 are characterized by the following steps: (a) detecting minimal levels of growth on media lacking histidine; and (b) detecting growth on media lacking histidine in the presence of weak Gal4-transcriptional activators. One of these two reporters, and most preferably the reporter displaying more sensitive response to activation is used for the yeast strain modifications described below.

Reporter 5: Reporter 5, which incorporates the LacZ gene, is characterized by detecting differential β-galactosidase activity in the presence of strong and weak transcriptional activators.

Reporter 6: Reporter 6, comprised of the LacZ gene under the transcriptional regulation of 8 LexA operator (8op) was integrated in place of the LYS2 gene in the genome of strain yVT87 creating strain yVT98.). Following integration, the reporter was determined to have the following properties; (1) the reporter conferred a LacZ+phenotype to the host yeast strain in the presence of a strong, LexA-fused, transcriptional activator; (2) the reporter conferred a LacZ+phenotype to the host yeast strain in the presence of a pair of interacting proteins, one expressed as a LexA fusion (p53) and the second fused to the B42 activation domain (Large T-antigen); and (3) the reporter did not display a LacZ+phenotype in the presence of LexA fusions that do not normally activate transcription.

(6) Creation and Characterization of Exemplary Host Yeast Strains

Construction of exemplary but non-limiting validator yeast-reporter strains is as follows.

YVT96: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT96, MATα ura3-52 his3-200 ade 2-101 ade5 lys2::GAL2-URA3 leu2-3, 112 trp1-901 tyr1-501 gal4D gal80Δ ade5::hisG by homologous recombination of Reporter 1 to the LYS2 locus. The integration is confirmed by PCR.

YVT97: The starting strain is YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 will be converted to yVT97, MATα ura3-52 his3::GAL1 or GAL7-HIS3 ade2-101 ade5 lys2-801 leu2-3, 112 trp1-901 tyr1-501 gal4Δ gal80Δ ade5::hisG by the steps of (a) converting from MATa to MATα via transient expression of the HO endonuclease, Methods in Enzymology Vol. 194:132-146 (1991) and (b) integrating either of Reporters 3 or 4 at the HIS3 locus via homologous recombination. The integration is confirmed by PCR.

YVT98: The starting strain was EGY48 (Estojak, J. Et al., 1995) MATα, ura3 his3 trp1 leu2::LexAop(x6)-LEU2. EGY48 was converted to strain yVT98 MATα ura3 his3 trp1 leu2::lexAop(x6)-LEU2 lys2::lexAop(8x or 2x)-LacZ by homologous recombination of Reporter 6 into the LYS2 locus.

YVT99: The starting strain was EGY48 (Estojak, J. Et al., 1995) MATα, ura3 his3 trp1 leu2::LexAop(x6)-LEU2. EGY48 was converted to strain yVT99 MATα ura3 his3 trp1 leu2::lexAop(x6)-LEU2 lys2::lexAop(8x or 2x)-URA3 by homologous recombination of Reporter 2 into the LYS2 locus and by switching the mating type from MATα to MATa via transient expression of the HO endonuclease.

YVT100: The starting strain was YM4271 (Liu, J. et al., 1993) MATa, ura3-52 his3-200 ade2-101 ade5 lys2-801 leu2-3, 112 trp1-901 tyr1-501 gal4Δ gal8Δ ade5::hisG. YM4271 was converted to yVT100, MATα ura3-52 his3-200 ade2-101 ade5 lys2::lexAop(8x or 2x)-URA3 leu2-3, 112 trp1-901 tyr-501 gal4Δ gal80Δ ade5::hisG by homologous recombination of Reporter 2 to the LYS2 locus. The integration was confirmed by PCR.

### EXAMPLE 3

### Identifying Physiologically Relevant Targets in a Melamona Cell Line

The invention can be applied to find targets of perturbagens that have been isolated using selections/screens in mammalian cells. As an example, perturbagen libraries are introduced by retroviral gene transfer into HS294T melanoma cells that contain a regulated p16 gene. The induction of this gene leads to cell cycle arrest and ultimately death caused by p16 overexpression. Cells that escape from p16-mediated arrest and death are recovered following this first phenotypic assay. The resident perturbagens are isolated by PCR amplification using primers specific to the perturbagen flanking DNA sequences.

Next, a first protein interaction assay isolates the endogenous cellular components that bound to the first set of perturbagens. The perturbagen sequences are cloned so as to produce BD Gal4 or LexA fusions in a yeast expression vector and introduced into haploid yeast. The yeast strain used for the initial two hybrid selection in the case of the Gal4 based system is, e.g., yVT97. Alternatively, the perturbagens are cloned into a LexA based system, and yeast strain yVT98 is used. The prey libraries are then co-transformed into yeast harboring the BD-perturbagen fusion constructs, and yeast cells expressing the selected reporter as a result of AD/BD interaction are selected. This first assay consists of an initial selection on either defined media lacking histidine (Gal4 system) or leucine (LexA system), followed by an optional secondary screen for prey-bait interaction that monitors resultant expression of the LacZ reporter. Plasmid DNA encoding candidate targets can be recovered individually from surviving yeast by standard procedures.

An alternate method for performing the initial protein interaction assay is also available. In this case the perturbagen sequences are expressed as either a LexA fusion protein in, e.g., yVT98 or as a GAL4 BD fusion protein in eg., yVT97. “Prey” libraries (cDNA clones expressed as fusion proteins with either the B42AD or the GAL4AD) are placed into yeast strains of the opposite mating type such as yVT99 (LexA system) or yVT96 (GAL4 system). Prey libraries and perturbagen sequences are then introduced into the same cell by standard mating procedures. Selection for prey clones that interact with perturbagens can then be performed by using any combination of the available markers (e.g LEU, URA, LacZ for the YVT98/99 combination and HIS, URA, LacZ for the yVT96/97 combination. Plasmid DNA encoding candidate targets can then be recovered from surviving yeast by standard procedures.

An optional step to remove some artifactual false positives prior to recovery of the DNA is performed in the following manner. Individual survivors of the first round can be pooled and induced to lose the perturbagen containing plasmid through growth in non-selective media and/or use of a negative selection. Yeast harboring only the candidate target-encoding plasmids will then be mated to strains yVT96 (Gal4) or yVT99 or yVT 100 (LexA) that harbor “false baits” such as the lamin protein. Selection for diploids can then be carried out in the presence of 5-FOA. In this manner only diploids are enriched for cells that will grow and form colonies. DNA from the therapeutic target cell line used in the phenotypic assay is then recovered by standard methods.

Next, another protein interaction assay is performed. The second round of two hybrid selections occurs between the putative therapeutic targets (endogenous molecules that bound to the first set of perturbagens) and a second, independent perturbagen library—e.g., a random-primed library of, e.g., human fetal brain mRNA, expressed as fusions with the Gal4 AD, or synthetic DNA encoding a peptide library. Generally these selections involve a mating between yeast strains harboring one or more of the candidate targets and yeast strains harboring the appropriate cDNA or peptide perturbagen probe libraries. Strains used are yVY96 and 97 in the case of the Gal4 system and yVT98 and either vVT99 or yVT100 in the case of the LexA system. Candidate targets may be subcloned to the binding domain side prior to these selections. These selections are carried out as in the first round, except that false positives will not be depleted following the selection.

For embodiments in which a peptide library is utilized as the second, independent perturbagen library, this second protein-protein interaction assay is performed as follows. The second round of two hybrid selection occurs between the candidate targets (obtained in the first two hybrid selection between the perturbagen sequences and “prey” cDNA libraries) and random peptide libraries. In one embodiment of this round of selection, the candidate targets, obtained as fusions with an activation domain (either GAL4 or B42), are subcloned such that they are expressed as DNA binding domain fusions (either LexA or GAL4). Yeast harboring the candidate target-BD fusions (yVT98 and yVT97 in the LexA and GAL4 systems, respectively) are mated to yeast strains of the opposite mating type (yVTs 99 and 96) that carry a GFP-peptide “prey” library (e.g., Abedi et al. (1998), incorporated herein by preference in its entirety). Selections for peptides that bind the various candidate targets are then performed using available markers, as described previously. False positives obtained in this round of selection would not need to be removed as in the prior selection.

Optionally, peptides that bind to candidate targets are identified without the requirement that the candidate targets be expressed as binding domain fusions. One advantage of such a strategy is that the need to subclone these target-encoding sequences from the AD fusion vector in which they were initially identified to a binding domain expression vector is eliminated. Furthermore, the possibility that they could be unusable as BD fusions due to self-activating properties is rendered moot. This type of selection is identical to the selection described elsewhere, except that the candidate targets are expressed as AD fusions and the GFP-peptide “prey” library sequences are expressed as a binding domain fusion. Self-activating peptide sequences are removed prior to the actual selection, using techniques described elsewhere herein.

In alternative embodiments utilizing libraries derived from either cDNA or gDNA, the set of candidate targets identified by the primary phenotypic probe sequences is tested against a second, independent random-primed library of, e.g., human fetal brain mRNA or gDNA, expressed as fusions with the Gal4 AD. The two-hybrid selections then are carried out as detailed above.

Next, a second phenotypic assay is performed as follows. The recovered peptide library or cDNA library sequences are recloned into a mammalian expression vector, e.g., a retroviral vector. These sequences are introduced once again into HS294T melanoma cells engineered with p16 and the cells are subjected to selection wherein escape from p16-mediated arrest and death is required. The cells that pass this test and form colonies are recovered and their resident perturbagen-encoding sequences isolated by PCR. These sequences are tested against the set of candidate targets in the same manner as described above, involving a selection on media lacking either histidine (Gal4) or leucine (LexA) and a secondary screen that monitors expression of the LacZ gene. A candidate target that binds to one of the confirmatory phenotypic probes is thus identified as a validated, physiologically relevant target.

### EXAMPLE 4

### Optional Steps for Improving the Efficiency of a Yeast Two-hybrid Protein Interaction Assay

In some cases it may be desirable to switch the candidate targets from the activation domain side to the binding domain side between the first and second rounds of two-hybrid selections. This can be accomplished in a number of ways that use standard practices of molecular biology including, but not limited to, PCR, subcloning and gap repair.

Also in some cases it may be desirable to remove self-activating sequences from two-hybrid libraries prior to a two hybrid selection. This is most important in the case of protein or peptide fusions with the Gal4 and LexA DNA binding domains as a large percentage of random sequences can activate transcription.

To remove self-activating sequences from DB-fusion libraries (e.g., cDNA fragment or peptide “prey” libraries) the following general methodology was performed. Yeast strain yVT99 was transformed with a pVT560-based (LexA) library. Of 5×10⁵ yeast carrying this library plated on media lacking leucine 55+/−6 yeast were able to divide and form colonies, indicating that roughly 0.014+/−0.005% of the peptides in this library were self-activating. To remove yeast expressing self-activating peptides from the library as a whole, 7×10⁶ yeast (0.5-fold coverage of the library) were plated on defined media lacking histidine (to select for the library plasmid) and containing 0.25% 5-FOA. Counting of yeast colonies formed on dilution plates indicated that plating of both the library containing yeast and yeast carrying a control plasmid on plates containing 5-FOA did not have a detrimental effect on yeast growth and division in general. Yeast carrying library plasmids were then recovered from the 5-FOA media and frozen in aliquots. Of ˜1×10⁶ yeast passaged over the 5-FOA and subsequently plated on defined media lacking leucine and uracil no yeast were able to divide and form colonies, indicating that the 5-FOA treatment completely eradicated self-activating sequences from the library population as a whole.

Similar negative selections can also be performed on binding domain-cDNA libraries in order to facilitate two hybrid selections involving candidate targets that self activate transcription.

### EXAMPLE 5

### Using Biochemical Methods to Detect Validated Protein-ligand Interactions

As an alternative to yeast two-hybrid protein interaction assays, it is possible to use affinity purification to identify endogenous proteins from therapeutic target cells that bind to perturbagen. The first step involves use of at least one perturbagen as an affinity reagent to select from a cell extract proteins that bind the perturbagen(s). This is performed with individual perturbagens, or alternatively, en masse with a collection of perturbagens. The perturbagens preferably have attached to them a label that permits the use of a generic binding matrix to attach them to a solid support. Examples include the FLAG epitope, HisTag, maltose-binding domain, glutathione-S-transferase, and others.

After incubation with the cell extract under conditions of salt, pH, etc. appropriate for binding and affinity purification. In many cases, conditions that reproduce physiological pH and salt levels in the cell are appropriate. In other cases, conditions that permit binding between the label or tag and an affinity matrix are demanded (e.g., conditions suitable for interaction between glutatione-S-transferase and its ligand, glutathione. These conditions can be gleaned from standard suppliers' instructions, or from standard molecular biology protocols. The perturbagen(s) and their attached cellular proteins are separated from the bulk of unbound cellular proteins by a series of routine washing steps designed to remove non-specifically bound proteins. The enriched complexes of bound proteins and perturbagen(s) are collected for analysis.

Next, one analyzes the protein(s) bound to a single or set of perturbagens. One particularly attractive method of doing so is to use recently-developed mass spectrometric methods. Mass spec instruments are commercially available and can be used in a variety of contexts to analyze macromolecules including proteins. In one version, the sample of perturbagen-bound proteins is first proteolyzed with a specific protease or collection of proteases, fractionated on a HPLC (high pressure liquid chromatography) column, and subjected to MALDI mass spec. From the peaks that are detected, charge/mass ratios are measured and amino acid composition of individual peptide fragments are inferred. The amino acid compositions can be compared against predicted fragments from a protein or translated DNA database. If matches are found, perturbagen-binding partners can be identified based on the match, typically with a high degree of confidence. The sum of all the database “hits” in principle defines the family of candidate perturbagen-binding proteins in the original sample.

The next step in the process requires identification of peptides or protein fragments that physically interact with individual members of the family of perturbagen-binding proteins. One biochemical strategy for isolation of such agents involves the use of expressed, purified protein using phage display. Full length cDNA encoding the above-identified binding partners can be constructed or obtained from commercial organizations. These clones can either be transferred into suitable expression constructs or used directly to produce in, e.g., E. coli a substantial quantity of the given protein. The protein can be purified by a variety of methods known in the art and used as the basis for phage display experiments. In these experiments, the purified protein is typically attached to a solid support and serves to select from a library of peptides displayed on the surface of phage a set of secondary candidate perturbagens.

Finally, one identifies the physiologically relevant binding partners. For example, the set of DNA fragments encoding these candidate confirmatory perturbagens can be cloned into a mammalian expression vector, e.g., and the entire population can be introduced into the assay originally used to isolate the primary perturbagens. Those secondary perturbagens that are recovered from the assay (i.e., that have physiological effects similar to the primary perturbagens) are derived from specific candidate targets; that is, they bind to specific candidate targets identified as above. The candidate targets that bind both primary and secondary perturbagens as judged by biochemical experiments are the physiologically relevant binding partners, i.e., the perturbagen targets in vivo.

### EXAMPLE 6

### Validation of an Endogenous Target in Yeast

As an example of the application of the invention to screening in yeast, a series of experiments led to identification of perturbagens that confer resistance to growth arrest caused by pheromone (Caponigro et al., 1998). One candidate target identified by this perturbagen screen was STE11p, the STE11 gene product (Id.). In order to validate the function of STE11p, i.e. to verify that STE11p is indeed a physiologically relevant target in yeast, the following experiment was performed.

The entire STE11 gene was cloned in frame with the LexA protein in the vector pLexA (Clontech). The LexA-STE11p expressed well, as judged by western blot analysis, and did not self-activate transcription when introduced into strain EGY48 (a precursor to strain yVT98). In order to identify peptides that bound to STE11p the following steps were performed. First, roughly 3×10⁶ members of a pVT592-based peptide library were co-transformed into yeast expressing the LexA-STE11p fusion protein. Second, peptides in the library that were able to bind to the LexA-Ste11p fusion were identified by selecting for yeast able to grow on defined media in the absence of leucine, and were also able to activate transcription of a separate LacZ reporter.

Sequence analysis indicated that approximately 68 different putative Ste11p binding peptides were obtained in the initial two hybrid selection. Further testing of a subset of these putative binders with a false bait (a LexA-p53 fusion protein) and the “real” bait (LexA-Ste11p) indicated that roughly half of the binders obtained in the selection were specific for Ste11p. In total, from a library of 3×10⁶ clones ˜37 (0.001% of total library clones) distinct Ste11p-binding peptides were obtained. Thus, GFP-scaffolded peptides were a good source of Ste11p-binders.

To identify peptides able to inhibit Ste11p in vivo the following experiment was performed. The entire set of putative Ste11p binders obtained in the initial selection were subcloned en masse into pVT27, which permitted their high expression from the galactose regulated GAL1 promoter (Abedi et. al 1998). This expression library of Ste11p binders was introduced into strain yVT12 and cells able to escape alpha-factor-induced cell cycle arrest identified as described in (Caponigro et. al 1998). Plasmid DNA was isolated from cells escaping this alpha-factor induced cell cycle arrest and re-tested in naïve yeast in order to establish linkage between the escape phenotype and individual peptide sequences. In total, two different peptides were found to confer resistance to alpha-factor mediated cell cycle arrest. Thus, this methodology provides a rapid and effective way to validate candidate targets.

This methodology may be further applied to identify and validate components of the pheromone response pathway. To find the unknown targets, the first set of perturbagens are expressed as fusions with either the LexA or Gal4 BD in yeast cells. These fusions are the “bait” and are tested for interaction with members of a prey library consisting of randomly sheared yeast genomic DNA (gDNA) cloned to encode fusions with the Gal4 AD on a yeast expression plasmid. The bait and prey libraries are examined together in haploid yeast cells following co-transformation. Selection for expression of any of a number of available markers, e.g., URA3+, defines a subset of prey sequences that interact physically with bait sequences. These are collected using PCR amplification or plasmid isolation.

The AD fused candidate targets can be used directly against a library of peptides (15 amino acids) displayed on a GFP scaffold that is fused to, e.g., the LexA BD. This prey library has been depleted of members that activate in the absence of a second physical interaction by negative selection against the URA3+phenotype. Peptides are isolated from cells surviving the two-hybrid selection between the AD-fused candidate targets and BD-peptide/GFP fusion constructs, and recloned into a galactose-regulated expression vector that contains GFP, capable of expressing peptides fused within the GFP scaffold.

The sublibrary of GFP-peptide fusions is reintroduced into yeast cells and yeast are identified that grow in the presence of pheromone and galactose. These yeast are further tested to ensure that their escape is galactose-dependent. Those that express peptides that confer resistance to pheromone are collected and used in a second focused two-hybrid assay to identify binding partners from the original set of candidate targets. The candidate targets from the original prey library which bind to any member of the second set of perturbagens are considered to be valid in vivo targets having physiological relevance that may be potentially used in, for example, development of anti-fungal agents, or alternatively may be extrapolated to human physiological pathways.

### EXAMPLE 7

### Validation of an Endogenous Target in Virally Infected Cells

Perturbagens can be used to identify points of vulnerability in the pathways involved in viral infection. These points of vulnerability may include viral proteins or cellular proteins required by the virus for productive infection.

As an example, adenovirus infects humans producing in some cases cold-like symptoms. To find adenovirus targets for antiinfective drugs, adenovirus was engineered to contain the GFP gene regulated by the CMV promoter (Adeno-GFP, Cat. No. AES0515, Quantum Biotechnologies, Montreal, PQ, Canada) Cells productively infected by this virus fluoresce bright green, and thus can be readily visualized or sorted by standard methods.

Epstein-Barr viral vectors containing the putative perturbagen encoding sequences were constructed as follows: GFP was mutated at codon 66 (Y66F) in order to eradicate fluorescence (“dead” GFP). Perturbagen-encoding sequences were then inserted into the dead GFP scaffold at the C-terminus. Two perturbagen libraries were constructed: the first library utilized synthetic peptides, the second utilized cDNA derived from human placenta polyA+mRNA.

The perturbagen constructs were transfected into human 293 cells using lipofection and allowed to express the perturbagen/dead GFP fusions for two days. These perturbagen-containing cells were then infected at a MOI of 10 with the recombinant adenovirus expressing fluorescent (“live”) GFP. In order to enrich the population for cells that are not productively infected with adenovirus, the cell population was trypsinized 36 hours after infection. Cells that do not subsequently re-adhere were removed by washing, when the cells were harvested at 48 hours. The cells were then sorted by flow cytometry. Those cells that were dim (i.e., exhibiting low fluorescence) were recovered by flow sorter and their resident perturbagen-encoding sequences are recovered by PCR.

After two cycles of reintroduction and infection, the perturbagens that confer resistance to adenovirus infection are identified and their encoding sequences are cloned into a BD vector. Validated, physiologically relevant targets are identified by pursuing the same steps as described in the previous example.

### EXAMPLE 8

### Exemplary High Throughput Procedures

(1) Picking Bacterial or Yeast Colonies

Numerous methods can be used to pick bacterial or yeast clones that contain individual library members. Colonies can be hand picked from agar plates using a sterile toothpick or eppendorf tip and inoculated into separate wells of a 96-, 384-(or larger) well plate containing media that will support bacterial growth (e.g. LB+Amp). More conveniently, simple automations that combine image processing and robotic techniques may be used to scan agar plates and pick individual colonies (Uber, D. C. et al. (1991) “Application of robotics and image processing to automated colony picking and arraying.” Biotechniques, 11(5) 642-647; Jones, P. et al. (1992) “Integration of image analysis and robotics into a fully automated colony picking and plate handling system.” Nucleic Acids Res. 20(17):4599-606). As a third option, the cell density of cultures containing members of a sublibrary can be diluted to a sufficiently low level such that only a single cell is likely to be present in a given volume of media. Pre-measured aliquots can then be pipetted into individual wells with a reasonable assurance that each well contains a single clone. Alternatively, automated, high-speed FACS machines (e.g. Cytomation Moflo) can be programmed to deliver single cells into individual wells. These methodologies, and others, can be utilized to isolate individual clones of the sublibrary.

(2) Isolation and Packaging of Plasmids

Isolation of each peptide-encoding retroviral vector from mini-bacterial cultures grown in, for instance, 96-or 384-well plates, can be achieved using procedures common to the art. As an example, high-throughput plasmid purification can be performed by combining automated procedures with standard anion-exchange technology (see, QIAwell Ultra BioRobot Kits and Bio-Robot 8000, Qiagen®). Highly infective retroviral supernatants may then be prepared by introducing each plasmid (along with the appropriate co-vectors, e.g. VSV-G envelope expression plasmid) into HS293 gp packaging cells similarly plated in either the 96-or 384 well format. Like the previously described plasmid purification procedures, transfection of the HS293 gp cells with retroviral DNA and collection of the resultant viral supernatants can be accomplished using common, “off-the-shelf” automated technologies (e.g. Sorval “Cytomat” and a Beckman “Multimek” instrumentation.)

(3) Bioassays

All of the bioassays described below can be performed using one or more components of high throughput, automated technologies. In high-throughput FACS assays, cells are detached from the 96- or 384-well plate using, for instance, trypsin, and analyzed directly in a fluorescent activated cell sorter (FACS) to determine the fraction of the population that expresses a fluorescent reporter gene that correlates with the phenotype of interest. Alternatively, in the GFA assay described herein, simple robotics are used to calculate the differences in numbers of colonies on plates that contain peptides that inhibit hsGFA, versus those that contain non-inhibiting, control peptides. In negative selection assays (designed to identify perturbagens that limit cell growth or induce cell death) a signal, such as fluorescence emission, is measured from each well using an automated CCD camera (MetaMorph®, Universal Imaging Corporation) or fluorescent plate reader (e.g.FluorTracker® Fluorescent Plate Reader, Stratagene). While the characteristics of stains (e.g. propidium iodide, Sytox® (Molecular Probes)) used in these procedures can vary greatly, the principles of the screen are, in general, similar. For instance, in a Sytox® stain, a monolayer of cells in a 96 well plate are directly exposed to the agent (10 nM-1uM) for 10-15 minutes. As Sytox® has affinity for DNA and is capable of staining the nucleus of membrane compromised cells, samples can then be illuminated with the proper wavelength of light (e.g. Ex504 nm for Sytox Green) and analyzed (Em 523) to determine the total number of dead cells, or, for instance, the ratio of living cells to dead cells.

### EXAMPLE 9

### High Throughput GFA Assay

The term “diabetes mellitus” describes a group of diseases characterized by high levels of blood glucose resulting from defects in insulin secretion, insulin action, or both. Clinically, Type I and Type II diabetes are the most prevalent forms of the disease.

One protein believed to have a role in Type II diabetes (also referred to herein as NIDDM) is glutamine:fructose-6-phosphate amidotransferase (also referred to as GFA or GFAT). In the following example, a unique set of methods are described to identify GFA binding peptides that disrupt the physiological function of the specific target, GFA. Such GFA binding peptides identified by these techniques are potentially useful as direct therapeutic agents in the treatment of patients afflicted with Type II diabetes. Alternatively, the GFA binding peptides may be used in conjunction with GFA to establish small molecule (competitive) screens designed to identify mimetics that are able to bind to GFA and displace the GFA binding peptide.

(1) General Background

GFA binding agents are isolated using the following exemplary procedures. In the first step, peptides exhibiting an affinity for the target, GFA, are isolated using one of a variety of techniques that are common to the art. These techniques include, but are not limited to, column chromatography, phage display, and high-throughput chip technologies (see, for example, Hentz, N. G. and Daunert, S. (1996) “Bifunctional fusion proteins of calmodulin and protein A as affinity ligands in protein purification and in the study of protein-protein interactions.” Anal Chem. 68(22):3939-44, Cwirla, S. E. et al. (1990) “Peptides on phage: a vast library of peptides for identifying ligands.” Proc Natl Acad Sci 87(16):6378-82, and HIP™ Chip/Profusion Technology, Phylos). Peptides with an affinity for the GFA target also may be isolated using two-hybrid procedures familiar to those of ordinary skill in the art (The yeast two-hybrid system, Oxford Univ. Press (1997), Bartel, Paul L. and Fields, Stanley, Ed.). Briefly, the GFA coding sequence is fused in-frame, to the C-terminus of, for instance, the Gal4 binding domain, and introduced into a standard yeast two-hybrid host cell (e.g. yVT 360). Subsequently, sequences encoding a random peptide library, fused to the C terminus of an activation domain (e.g. VP16 AD) are transformed into the host carrying the GFA-BD target and screened for GFA interacting peptides by plating the cells on the appropriate selective.

In the second step of these procedures, GFA-binding peptides are tested for disruptive activity. Briefly, the polynucleotide sequences encoding each GFA-binding peptide are transformed into haploid yeast cells that are deficient in the yeast homologue of GFA (ygfa1−) yet contain a complementary human version of the enzyme (i.e. ygfa1⁻/hsGFA⁺). In yeast, glutamine:fructose-6-phosphate amidotransferase is required for synthesis and maintenance of the cell wall, and is therefore essential for viability and growth. As it has been demonstrated that human GFA1 complements knockout mutations in the yeast GFA gene, it is possible, through sporulation of yGFA1⁺/ygfa1⁻diploids transformed with human GFA1, to obtain viable haploid progeny that are genotypically gfa1/hsGFA1⁺. Using this novel strain, each GFA binding peptide is tested for disruptive activity by introducing each peptide into the gfa1⁻/hsGFA1 haploid strain and tested for viability. Peptides that exhibit an affinity for GFA but fail to disrupt the activity of the human protein are viable. In contrast, peptides that bind GFA and disrupt enzymatic activity are non-viable or exhibit alterations in growth rate as demonstrated by the number or size of plated colonies, or the OD 600 measurement of liquid cultures. Inhibitory peptides can be propagated in yeast in the presence of glucosamine and assayed for activity in the absence of glucosamine.

(2) GFA Two Hybrid Materials and Methods

Preparation of various yeast two-hybrid assay components—e.g., bait constructs, prey constructs, and host cells—are familiar to the art. The following are exemplary, non-limiting examples of such components.

YEAST VECTORS: The target and perturbagen libraries are deployed as bait/prey libraries in appropriate bait and prey fusion constructs. One exemplary binding domain vector is pVT701 (pGBKT7,Clontech) which includes the yeast GAL4 DNA binding protein fused to a multiple cloning site. This vector also contains the yeast TRP1 gene and the kanamycin resistance gene (KanR) for selection in yeast and bacteria, respectively.

Suitable activation domain vectors for peptide perturbagens or peptide prey libraries include pVT2512, pVT2018, pVT2025, pVT2019, pVT562 and pVT592. An exemplary vector for peptide prey libraries expressed as AD fusions is pVT2019, which has a C-terminal peptide fused to the activation domain VP16. The vectors pVT2019 was constructed as follows: a stuffer fragment (amplified from pVT404 with primers oVT3302 (5′ CCC CGG ATC CCG GGC CGA GGC GGC CCC GCG G) and oVT3349 (CCC CCC TCG AGG CGG CCG CGG CCA TTA TGG CC)) was inserted into the BamHI/NotI site of pVP16. SfiI was used to ligate annealed oligos encoding peptide fragments into the library vector. As a result of these procedures, each peptide is inserted in-frame with the VP-16 activation domain. Said constructs can subsequently be transformed into bacteria (in the presence of glucosamine) and then transferred into an appropriate yeast strain, for example yVT360.

Peptide Libraries: As one non-limiting example of how a peptide library is constructed, random 45 bp oligonucleotides flanked with a constant sequence and the appropriate restriction sites (SfiI) are cloned into the compatible sites of pVT2019. Specifically, pVT2019 was digested with SfiI and the vector fragment (minus the “stuffer”) was band isolated on an agarose gel. The splint cloning strategy (Abedi et al supra) was then used to directionally clone random-peptide encoding DNA fragments into pVT2019. To accomplish this, fifteen picomoles of Aptamer 3305 (5′ CGG CCA CGC TGG A) was mixed with Aptamer 3307 (5′ TGG CCT ATT TAT TCA (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN TCC AGC GTG GCC GCC T) and Aptamer 3352 (5′ phosphate TGA ATA AAT AGG CCATAA)) in a molar ratio of 1:50:50 and annealed in 20 mM Tris-HCl, pH 7.5, 2 mM MgCl₂, 50 mM NaCl by heating to 70° C. for 5 minutes. The solution was then allowed to cool to room temperature and ligated into the SfiI cut pVT 2019 vector using T4 DNA Ligase (NEB). As a result of these manipulations, a constant region plus a biased-random fifteen amino acid sequence is fused to the C-terminus of VP16 AD. The library was then transformed into E. coli (DH10B, Gibco) by electroporation and spread on plates that contained i) the selective marker, ampicillin, and ii) glucosamine (5 mg/ml, Sigma). These procedures yielded a pVT2019 library size of approximately 4×10⁷ unique colonies. Plasmid DNA was then recovered using a Qiagen Maxiprep and used for direct transformation screening in yeast strain yVT360 harboring GALBD-GFA plasmid (Lithium Acetate TRAFO method (described previously). Transformants are then spread on SD (−Leu, −Trp, +glucosamine) to select for yeast that harbor the both the GALBD-GFA construct and a member of the peptide library.

hsGFA Gal4 BD Construct: The hsGFA clone was released from hGFA-Bluescript (generously donated by Dr. Don McClain, U. of Utah) using NdeI/BamHI. The fragment was then inserted into the MCS of pGBKT7 (Clontech). As a result of these procedures, the hsGFA1 clone is fused in-frame with the C-terminus of the Gal4 DNA binding domain. This fusion construct is associated with a 2μ plasmid carrying a TRP+ selectable marker is referred to herein as pVT1374 or GALBD-hsGFA1.

Identifying hsGFA1 Binding Peptides: To identify hsGFA1-binding peptides, the pGALBD-hsGFA1 construct is introduced into the yVT360 yeast cells. The resultant strain is then transformed with the pVT2019 peptide library. Briefly, an overnight culture of yVT360 harboring pGALBD-hsGFA1 cells is diluted into 50 mls of pre-warmed media (SD, −His, +glucosamine) at 5×10⁶ cells per ml. The culture is then incubated at 30° C. on a shaker (200 rpm) until the cell density reaches 2×10⁷ cells/ml. Cells are then harvested by centrifugation (3000×g, 5 min) and washed/centrifuged 1× with dH₂0. Subsequently, the cell pellet is washed in 1.0 ml of 100 mM LiAc, spun, and then resuspended in 400 ul of 100 mM LiAc. This procedure is scaled up for library transformation. Fifty microliter samples of the cell suspension are then alliquoted into microcentrifuge tubes and spun. The overlying layer of LiAc is gently removed and the following ingredients are added in order: 240 μl of 50% PEG (w/v), 36 μl 1M LiAc, 50 μl of 2.0 mg/ml salmon sperm DNA (boiled, denatured), 0.1-10.0 μg of pVT 2019-library DNA, and dH₂0 (to a total volume of 360 μl). The tubes are then vortexed to resuspend the pellet and incubated at 30° C. for 30 minutes. Each sample is then heat shocked (42° C., 30 min) and then centrifuged briefly (15 seconds) in an eppendorf (6-8,000 rpm). Following the removal of the transformation mix, the cells are resuspended in 1 ml of sterile water and plated (2-200 μl) onto selective plates (e.g. −Trp, −Leu, +glucosamine). Colonies carrying both constructs (pACT2-hsGFA1, and a member of the pVT2019 peptide library) are then pooled and replated on SD, −Trp, −Leu, −His, −Ade, +1 mM 3-AT, +glucosamine plates to identify clones that carry a peptide that has affinity for the hsGFA1 gene product. Individual colonies are then picked (by hand or automated procedures) and the DNA encoding the GFA-binding peptide is recovered by either PCR or plasmid rescue techniques that are common to the art.

(3) Testing hsGFA1-Binding Peptides for Perturbagen Activity

In order to test the GFA1 binding peptides for the ability to disrupt GFA enzymatic activity, it was necessary to develop a bioassay designed to detect loss of GFA function. One non-limiting whole cell assay identifies perturbagens on their ability to knock out human GFA activity in a haploid yeast containing one knockout copy of the yeast GFA homologue (yGFA1−) and one intact copy of the human complement (hsGFA1).

Complementation of GFA1⁻With hsGFA1: To test whether hsGFA1 was capable of complementing a knockout mutation in yGFA1, the cDNA encoding the human GFA1 gene was cloned into a yeast expression vector under the control of galactose-inducible promoter (pGal-GFA1, LEU2) using techniques that are common to the art. Under this configuration, hsGFA1 expression is regulated by pGAL. In the presence of glucose, hsGFA1 expression is off. In contrast, when glucose is absent and cells are grown in the presence of galactose, hsGFA1 expression is turned on. pGal-GFA1 (or a control vector, pVT11, LEU2, pGal-GFP) were then introduced into gfa1 heterozygous diploid yeast (YVT367 BY4743-YKL104C, Mata/Mat α his3 delta1/his3 delta 1, leu2 delta0/leu2 delta0, lys2 delta0/+, met15delta0/+, ura3delta0/ura3/delta0 gfa1::KAN/+, GFA1 knockout mutants were obtained from ATCC, ATCC #4024954). The transformants carrying the hsGFA1 construct (i.e. YVT367+pGAL−huGFA, LEU2) were then made haploid by sporulation and germinated on YEPD media containing glucosamine (5 mg/ml) to supplement the enzyme deficiency.

To test whether hsGFA1 was able to complement the loss of the yeast homologue, cells were grown on agarose plates containing galactose, but lacking the glucosamine supplement. Results show that in the absence of glucosamine, gfa1 null haploids (carrying a plasmid containing huGFA1 under the control of the GAL1 promoter) were unable to grow in a glucose medium (see Table 2). In contrast, when this strain was transferred to a galactose containing, glucosamine deficient substrate, the strain grew well. A control plasmid (pVT11) was not able to rescue the gfa1 null lethal when grown on galactose. These results indicate that human GFA1 can complement the lethality of the yeast gfa1 deletion mutant.

TABLE 2
Random spore analysis

| | | | Neo^{R}, | Neo^{R}, |
|---|---|---|---|---|
| | No. of | | YEPD−, | Leu gluc + |
| Mating | spores | Neo^{R}/Neo^{S} | Leu gluc+ | Leu Gal+ |
| BY4743-YKL104C | 26 | 13/13 | 5 | 0 |
| [pVT11] | | | | |
| BY4743-YKL104C | 52 | 21/31 | 7 | 6+ |
| [pVTGAL-GFA1] | | | | |

Screen of GFA Binding Peptides for GFA Perturbagens: To identify which of the GFA binding peptides are capable of acting as perturbagens, (i.e. are capable of inhibiting GFA activity) each construct is individually transformed into the hsGFA1/ygfa1⁻haploid line and tested for the ability to alter fitness. Specifically, hsGFA1/ygfa1⁻haploid cells grown in liquid culture supplemented with glucosamine are transformed with individual vector constructs encoding hsGFA1 binding peptides (HIS based vectors) and plated on selective plates (SD, −Leu, −His, +glucosamine). As a control, the parent vector (without an associated peptide) is transformed into ygfa1−/hsGFA1. Subsequently, equal numbers of cells from both the control and test samples are transferred to SD, −His, −Leu, −glucosamine, +galactose plates to examine for viability. Analysis of the number and size of colonies and growth rate is performed to determine which of the hsGFA binding peptides act to block GFA action.

### EXAMPLE 10

### High Throughput HPV-E6 Assay

Large proportions of the US and global populations (25-50%) are infected with a variety of human papilloma viruses (HPVs). High-risk HPV infection, generally types 16 and 18, are associated with over 90% of the cervical carcinomas and smaller proportions (roughly 20%) of other specific carcinomas. Extensive study has identified two gene products, E6 and E7, to be primarily responsible for HPV-related transformations. The following example describes the identification of peptides that demonstrate an affinity for the HPV E6 protein and which disrupt its physiological activity.

(1) Creation of Scaffolded Peptide Libraries

To identify peptides that bind to the HPV18 and HPV16 E6 proteins, a random peptide library was created and fused to the C-terminus of the VP-16 activation domain coding sequence. Briefly, a 15-mer of randomized nucleotides flanked on either side by constant regions were inserted into the SfiI site of a modified version of the pVT2025 vector (2 plasmid, Adh−NLS−VP16, LEU+) using the splint ligation procedures (see, for example, Abedi M. R. et al., (1998) “Green fluorescent protein as a scaffold for intracellular presentation of peptides.” N.A.R. 26(2): 623-630, Oligos 5′→3′: oVT3305 5′ CGGCCACGCTGGA, oVT3352 5′ TGAATAAATA GGCCATTA, and OVT 3307 5′ TCCGCCGGTGCGACCT(NNV15)ACTTA TTTATCCGGT). As a result of these procedures, the peptides were fused to the C-terminus of the activation domain of VP16. Library members (˜3.3×10⁶ in all) were then transformed into bacteria (DH5α, Gibco, BRL), grown in liquid culture under selective conditions (e.g. 100 ug Amp/ml), and prepared for transformation into yeast (Qiagen MaxiPrep) to identify peptide binders of the E6 proteins. To prepare for two hybrid procedures, the library was transformed into yVT69 (obtained from Clontech (Y187), yVT69=Y187=mat α, ura3-52, his3-200, ade2-101, trp1-901, leu2-3, 112, gal4 , met⁻, gal80 , URA3::GAL1_{UAS}−GAL1_{TATA}−lacZ) using standard techniques (LiAc TRAFO Method) and plated on SD, −Leu agar plates.

(2) Isolation of E6.

A HPV16 E6 clone was obtained (gift of Denise Galloway), the gene encoding E6 amplified and then cloned into pVT701. HPV18E6 proteins were isolated by PCR amplification from genomic DNA taken from an in-house HeLa cell stock (Oligos: E6, oVT 3391 (5′ GGG GGGAATTCTTATACTTGTGTT TCTCTGCGTCGTTGGAGTCG), oVT 3390 (5′ GG GGGCATATGATGG CGCGCTTTGAGGATCCAACACGGCG). Subsequently, the PCR product was cloned into the NdeI/EcoRI sites of pVT2014 (Kan^{R}, TRP+), resulting in a fusion of the E6 encoding sequence to the C-terminus of the Gal4 DNA binding domain. Upon confirming the E6 DNA sequence and the junction linking E6 with the BD, each construct was individually transformed into yeast yVT360 (obtained from Clontech, yVT360=AH109=mat a, trp1-901, leu2-3,112, ura3-52, his3-200, gal4 , gal 80 , LYS2::GAL1_{UAS}−GAL1_{TATA}−HIS3, GAL2_{UAS}−GAL2_{TATA}−ADE2, URA3:MEL1_{UAS}−MEL1_{TATA}−lacZ), plated on selective agar (SD−Trp,) and selected for transformants that carry an episomal copy of the plasmid.

(3) Identifying Peptide binders of HPV E6.

To identify peptides that demonstrate an affinity for HPV16 E6, a single colony of yVT360 expressing the target plasmid was grown in liquid media (SD−Trp), and then mated to yVT69 containing the pVT2025 random peptide library. Briefly, this was accomplished by mixing 2×10⁷ log-phase yVT360-GAL4BD-E6 cells with 4×10⁷ log phase yVT69 cells containing the GAL4-AD-peptide library in YEPD at a density of 10⁶ cells per ml. The culture, containing both “a” and “α” mating types, was then allowed to sit at room temperature (no shaking) for twenty-four hours before being centrifuged, resuspended in a small volume, and plated out on Leu⁻ Trp⁻ minimal media plates (48 hrs, 30° C.) to select for diploids. The surviving colonies (Leu⁺ Trp⁺) were subsequently pooled and re-plated on selective dropout plates designed to identify peptide binders (Leu⁻, Trp⁻, His⁻+2.5 mM 3-AT). Individual colonies were then collected, expanded in liquid culture, and processed to determine the sequence of the E6-binding peptides (ABI sequencer). Clones falling into at least ten complementation groups based on sequence (see Table 3) were obtained. Several of these peptides exhibited significant homology to SiHa growth inhibitors that have previously been shown to exhibit affinity for the E6 peptide (see, Butz, K. et al. (2000) “Induction of apoptosis in human papilloma virus positive cancer cells by peptide aptamers targeting the viral E6 oncoprotein.” Proc Natl Acad Sci USA 6;97(12):6693-7).

TABLE 3
SEQUENCES OF E6 PEPTIDE BINDERS

| Seq # | Copies | Sequence |
|---|---|---|
| 1 | 21 copies | LLVITIWQLWDEMLS |
| 2 | 6 copies | IDYSTAMNLLDSLLS |
| 3 | 3 copies | QTMNHALDVLYCLLG |
| 4 | 3 copies | ILRICLTCWIGFSS |
| 5 | 3 copies | LSKGAFILLDMLLGA |
| 6 | 2 copies | HDNFLELALEVLDPSLE |
| 7 | 2 copies | TLGWVTVFERLLGCD |
| 8 | 1 copy | DADVPSNP? |
| 9 | 1 copy | FCNAA?ASNNSL??? |
| 10 | 1 copy | APDLWYEIWDIILGK |
| 11 | 1 copy | KCCLCALLALPPPPSE |
| 12 | 1 copy | ADLHCCCLTCRIEAL |

(4) Peptide Competition Assay

To determine whether two peptides recognize the same epitope on the E6 protein, competition assays based on the yeast two-hybrid technology are performed. To accomplish this, yVT 360 cells containing the Gal4 BD-E6 fusion protein (pVT 2014, Trp+) are mated to yVT69 cells expressing i) the first E6 binding peptide (e.g. Seq #1) fused to the VP16AD (pVT2025, Leu⁺) and either ii) a control plasmid (His+) expressing a neutral scaffold (e.g. GFP) or iii) the control plasmid containing a second peptide (e.g. Sequence #2) fused to the C-terminus of the neutral GFP scaffold, or iv) an unscaffolded version of peptide #2 expressed by the same control plasmid. Diploids are selected by plating on SD, His-, Leu-, Trp- dropout plates. To determine whether the two peptides compete for the same E6 epitope, equal numbers of cells containing either the control plasmid or the suspected competitive peptide are allowed to grow on SD, His-, Leu-, Trp-, Ade- plates and the resultant number of colonies are compared. A matrix in which pair-wise competition between all twelve of the peptides is assessed allows mapping of the epitopes on the E6 surface and subdivision of the E6-binding peptides into functional complementation groups.

(5) Development of an E6 Sensitive Reporter Line

To test each E6-binding peptide for its ability to inhibit the action of E6, a TransFACS assay, based on the hTert promoter, is developed. The hTert promoter (P_{hTERT}) sequence (Horikawa, I. et. al. (1999) “Cloning and Characterization of the Promoter Region of Human Telomerase Reverse Transcriptase Gene.” Cancer Research. 59:826-830; Cong, Y -S et al. ( 1999) “The human telomerase catalytic subunit hTERT: organization of the gene and characterization of the promoter.” Human Molecular Genetics. 8:137-142. Takakura, M. et al. (1999) “Cloning of Human Telomerase Catalytic Subunit (hTERT) Gene Promoter and Identification of Proximal Core Promoter Sequences Essential for Transcriptional Activation in Immortalized and Cancer Cells.” Cancer Research. 59:551-557) was PCR amplified from human genomic DNA (Promega Human Genomic DNA, cat. no. G152A 4829402) using oligos OVT 1510 (5′ TAATCTTCTGCTTCCATTTCTTCTCTTCCCTC), and OVT 1561 (5′ GGCGGA AGGAGGGGGCGGCGGGGGGCGG). The resulting 1.59 kB fragment was TA cloned into PCR2.1 (Invitrogen), generating pVT816. Using primers OVT 1513 (5′ GGGGG GATCCTAATCTTCTGCTTCCATTTCTTCTCTTCCCTC) and OVT 1514 (5′ GGGGAAGCTTCGCGGGGGTGGCCGGGGCCAGGGC TTCCCACGGTG), the P_{hTERT} promoter was then PCR amplified from pVT 816, digested with BamHI/HindIII and ligated into the equivalent sites of the puromycin based vector, pVT807, to generate pVT 817. As a result of these procedures, PhTERT is positioned upstream of a GFP coding sequence. The pVT817 construct was then transformed into E. coli (DH10B, Gibco) by electroporation and plated on agar plates containing ampicillin. AMP^{R} colonies were then picked and the plasmid sequences contained within were isolated and sequenced to ensure no errors were introduced into the sequence as a result of PCR.

Two additional reporter constructs were prepared using pVT817. In the first, pVT 817 was digested with Avr II and BamHI, filled with Klenow Fragment, and self ligated to generate pVT 818. This truncated construct is shorter than the pVT817 construct but contains the major promoter element (bp-1-bp-333). In a second derivation of pVT817, the initial GFP was replaced with a destabilized GFP to create pVT 819. This was accomplished by digesting the pd2GFP-N1 vector (Clontech) with BamHI/XbaI, filling with Klenow Fragment, and ligating this into pVT818 that has been digested with ClaI and filled with Klenow.

Next, a clonal cell line in which the P_{hTERT}-GFP reporter is modulated by the HPV-E6 protein is identified. To accomplish this, hTERT reporter constructs are packaged in 293 gp cells (using standard techniques) and then introduced into E6-immortalized cells (HMEC, Clonetics). Following the isolation of stable inserts (puromycin selection, 3 days) the sensitivity of individual clones to the presence of HPV-E6 protein is determined by i) splitting the cultures, ii) growing one half of the cultures under conditions that allow loss of the E6 encoding retroviral insert, and iii) comparing the level of fluorescence of each P_{hTERT}-GFP clone in the presence and absence of E6. Clones that exhibit an E6-dependent increase in overall GFP expression are expanded and used in a TransFACS assay to determine which of the TBA's disrupt the interaction of E6.

(6) E6 FACS Assay

Clones that show an E6-dependent increase in overall GFP expression are used in subsequent bio-assays to determine which of the E6-binding peptides disrupt the action of E6. Specifically, each scaffolded E6-binding peptide is introduced individually into E6 immortalized cells harboring the hTERT-GFP reporter. After five days in culture, adherent cells are trypsinized (to separate them from the solid support) and analyzed on a FACS machine to determine which of the peptides cause all (or some fraction) of the population to exhibit decreased levels of fluorescence. In addition, the effect of each peptide is tested in a secondary assay by examining i) its effects on endogenous hTERT expression levels (Northern Blot analysis) ii) the ability of the peptide to block immortalization of HMEC cells by E6, and/or iii) the ability of the peptide to prevent proliferation of SiHa cells (HTB-35, ATCC).

### EXAMPLE 11

### Finding Peptide Binders to Subclasses of Proteins

The invention can be applied to examine groups or classes of proteins (e.g. kinases, phosphatases, GTP binding proteins, G-coupled protein receptors) in a single assay. Thus, for instance, one may evaluate a family of proteins that share a particular biochemical trait, for instance, all or substantially all human phosphatases, or some selected subset thereof, and identify a diverse class of peptides that exhibit affinity for one or more of the members of the selected family or subset. These peptides are then placed, en masse, into one or more bioassays, and tested to determine which member(s) induces a desired phenotype.

(1) Isolation of Peptide Perturbagens Directed Against Human Phosphatases

As one non-limiting example, phosphatase binding peptides are cycled through multiple rounds of a floater assay (see U.S. Ser. No. 09/504,132, “Methods for identifying agents that cause a lethal phenotype, and agents thereof,” the contents of which is incorporated by reference herein) to determine which of the agents induce cell death. Alternatively, the polynucleotide sequences encoding the peptides are introduced into HS294T melanoma cells that contain a regulated p16 gene. In this assay, one assesses whether any of the phosphatase binding peptides enable the cells to escape the cell death brought on by p16-mediated arrest. Subsequent recovery of such agent(s) and identification of the corresponding phosphatase partner by the methods described elsewhere herein enables one to then perform competition or displacement assays designed to identify small molecule mimetics capable of replacing the peptide(s) of interest.

(2) Isolation of Peptide Perturbagens Directed Against Human Kinases

As another non-limiting example of these procedures, a description of how these techniques could be applied to human protein kinases is now described.

A collection of human kinase cDNAs identified on the basis of sequence homology and/or the enzymatic activity of the respective translated protein, are fused in-frame with the binding domain of the Gal4 protein (pGBKT7, Clontech). Following the introduction of these constructs into yVT 360, each line is then transformed with the pVT2019 peptide library encoding a constant region plus a biased-random fifteen amino acid sequence fused to the C-terminus of VP16 (see above). Cells containing both constructs are subsequently isolated by plating the diploid yeast onto selective agar plates (e.g. −Trp, −Leu). Colonies carrying both constructs are then pooled and replated on SD, −Trp, −Leu, −His, −Ade, +1 mM 3-AT plates to identify clones that carry a peptide that has affinity for the kinase gene product. Individual colonies are then picked (either by hand or automated procedures) and the DNA encoding the kinase-binding peptide is recovered by either PCR or plasmid rescue techniques that are common to the art. The polynucleotide sequences encoding each agent is then PCR amplified (using flanking oligonucleotide primers), and cloned into a retroviral vector that constitutively expresses the peptide as a C-terminal fusion of the ZsGreen scaffold protein (pVT 2038).

To determine which of the kinase binding peptides are capable of inducing cell death in colorectal cancer cells, the retroviral constructs encoding the scaffolded kinase binding peptides are tested (either individually or en masse) in the HT29 floater assay (U.S. Ser. No. 09/504,132, supra). One non-limiting assay protocol is as follows: On Day 0, twenty T175 flasks are seeded with 2.2×10⁶ HT29 cells/flask in McCoy's 5A media (Gibco BRL) modified with 10% FBS. On Day 1, each flask is infected (4 μg/ml polybrene, 50% volume viral supernatant) with a retroviral supernatant containing the KBP encoding constructs. On Day 2 the media is changed and on Days 3 and 5, the floater cell populations are collected and combined. Genomic DNA is then prepared (QIAamp kit, Qiagen) and the polynucleotide sequences encoding each KBP are recovered using PCR. The kinase binding sequences are then re-cloned into the original retroviral vector, re-packaged in 293 gp cells, and recycled through the assay to enrich for sequences that induce cell death in HT29 cells.

### EXAMPLE 12

### Screening Genomes for Bioactive Peptides

A broader application of the previously described procedures focuses on developing peptide binders to all the proteins encoded in a given organism's genome. Large peptide libraries having constituents with affinity to all of the ORFs in a single organism can be tested, en masse, for phenotypes relevant to the focus of study. Thus, for example, target-binding peptides developed against all of the open reading frames of, for instance, influenza (or any other pathogen) can be tested in bioassays designed to identify agents capable of inhibiting viral replication. Similarly, peptide libraries having affinity for all or substantially all of the proteins encoded in the human genome can be screened for a wide range of relevant phenotypes.

In some cases, it may be desirable to isolate peptides that induce their phenotypes on unique cell types (e.g. isolating agents that induce cell death in cancer cells, but not in normal, wildtype, cells). Similarly, one may find peptides that induce differentiation in one cell type (for instance, HL60 promylocytic cells changing fate to pre-granulocytes) but not in others. Alternatively, it may be of interest to identify peptides that act on a broad range of cell types. Thus, for instance, it may desirable to identify agents that, e.g., i) induce apoptosis in all cells exhibiting multidrug resistance, or ii) increase glucose metabolism in both fat and muscle tissue.

In some cases, the genomes from which the encoded proteins are derived are taken from normal, wildtype cells. Alternatively, the starting material from which peptide binding libraries are derived can be isolated from cells that already exhibit well-defined genetic alterations (e.g. myc⁻, ras⁻, p57⁻). Regardless of the source of the starting material, recovery of bioactive peptide agents and identification of their target partner by the methods described elsewhere herein provide researchers with the opportunity to develop small molecule mimetics using standard displacement assays.

As one non-limiting example of how these procedures, a description of how these techniques could be applied to peptides developed against the proteins of the human genome is described.

(1) Isolation of Peptide Perturbagens from Genome Derived Libraries

In one non-limiting example, peptides having affinity against the products of all the open reading frames of the human genome are screened for agents that induce cell death in T47D metastatic mammary epithelial cells. In the initial steps, cDNAs derived from the T47D line are fused in-frame with the binding domain of the Gal4 protein (pGBKT7, Clontech) by gap repair or direct cloning and transformed into yVT360. In cases where the targets of interest are self-activators, they are cloned into the activation domain in-frame with VP16 and under the control of the inducible GAL1 promoter. In the case where a particular target is found to be a self-activator, a BD domain library is substituted for the previously described AD peptide library.

Specifically, fifteen picomoles of Aptamer 3305 (5′ CGG CCA CGC TGG A) is mixed with Aptamer 3307 (5′ TGG CCT ATT TAT TCA (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN (G/A/C)NN TCC AGC GTG GCC GCC T) and Aptamer 3352 (5′ phosphate TGA ATA AAT AGG CCATAA)) in a molar ratio of 1:50:50 and annealed in 20 mM Tris-HCl, pH 7.5, 2 mM MgCl₂, 50 mM NaCl by heating to 70° C. for 5 minutes. The solution is then allowed to cool to room temperature and is igated to the SfiI cut pVT 2543 vector (CEN, HIS3, Adh-LEXNLS) using T4 DNA Ligase (NEB). As a result of these manipulations, a constant region plus a biased-random fifteen amino acid sequence fuses to the C-terminus of LEXDB (the LexA binding domain). The library is then transformed into E. coli (DH10B, Gibco) by electroporation and is spread on plates that contained the selective marker, ampicillin.

In the next step, the library is distributed into the individual wells of a 96 well plate (i.e. to “array” the library). To accomplish this, electroporated DH10B cells are spread onto LB+KAN plates such that 1-2 million transformants are obtained per plate. The bacteria on each plate are then collected and placed into separate 15 ml conical tubes. The transformants in each conical tube are aliquoted to multiple 96 “deep well” dishes. Specifically, six milliliters of a bacterial-library suspension from one transformation plate is dispensed into the same well position (i.e. A1) in multiple separate deep well dishes. Similarly, the bacterial suspension from a separate transformation plate is dispensed into a separate well position (i.e. A2). This process is continued until all the wells in the 96 well plates contained bacteria from 96 distinct transformation plates. Glycerol is then added to two of the deep well dishes, and these are then stored at −80 for future amplification. The bacteria from the other dishes are spun down and the DNA is extracted from the pellet using standard molecular biological techniques. Subsequently, the DNA from each well is transformed into yeast (yVT99), keeping each well separate (Trafo method). This procedure is expected to yield a library size of approximately 2.5×10⁸ unique colonies distributed into a normal array. The transformants are then culled for self-activators (using FOA or other techniques, e.g. 5-fluoroanthranilic acid, see Toyn, J. H. (2000) “A counterselection for the tryptophan pathway in yeast: 5-fluoroanthranilic acid resistance.” Yeast 16(6):553-60.) and placed back into a 96 well format.

Using state of the art, high throughput technologies such as those described elsewhere herein, yeast cells (yVT87) carrying an individual target molecule and harboring a Zsgreen based reporter vector (Clontech) are mated (in a 96 well format, YEPD) to approximately 108 different members of the yVT99 strain carrying the peptide library (˜10⁶ peptide binders per well). The YEPD media is then replaced with a diploid selection media and cells are grown to late log phase. Subsequently, peptides having affinity to any given target are selected by transferring a portion of the culture into SD, −Ura, −Leu, −His, −Trp+HYG media. Interaction of the target with a given peptide is then detected by assaying for expression of the reporter. This may be accomplished by, for instance, measuring the expression of Zsgreen or growth in selective media. Plasmid DNA from positive wells is then i) purified, ii) cloned into an appropriate retroviral vector, iii) packaged in 293 gp cells, and iv) infected into T47D cells, grown in a 96 well format. At a defined period of time after introduction of the peptide into the T47D cell line, cells are stained with Cytox (Molecular Probes), illuminated with an excitatory wavelength of light and analyzed on a plate reader. Peptides that inhibit cell growth or are cytotoxic are then isolated by comparing the number of dead cells in test wells to wells that have been infected with a control plasmid.

The above examples are provided to illustrate the invention but not to limit its scope. Other variants of the invention will be readily apparent to one of ordinary skill in the art and encompassed by the appended claims. All publications, patents, and patent applications cited herein are hereby incorporated by reference.

What is claimed is:

1. A method for identifying a physiologically relevant target molecule that correlates to a phenotype of interest, comprising the steps of:
(a) determining a first protein-ligand interaction between a pool of therapeutic target candidates and a putative perturbagen library;
(b) isolating members of said putative perturbagen library that bind to members of said pool of therapeutic target candidates;
(c) introducing said isolated members into a host cell population;
(d) performing a phenotypic assay with said population in order to identify a sublibrary of physiologically relevant perturbagens;
(e) determining a second protein-ligand interaction between said sublibrary of physiologically relevant perturbagens and said pool of therapeutic target candidates; and
(f) identifying individual protein-ligand pairs between members of said sublibrary of physiologically relevant perturbagens and members of said pool of therapeutic target candidates, wherein an interaction of a physiologically relevant perturbagen with a therapeutic target candidate identifies said therapeutic target candidate as a physiologically relevant target molecule that correlates to a phenotype of interest.

2. A method for identifying a physiologically relevant target molecule that correlates to a phenotype of interest, comprising the steps of:
(a) determining a first protein-ligand interaction between a single therapeutic target candidate and a putative perturbagen library;
(b) isolating members of said putative perturbagen library that bind to said therapeutic target candidate;
(c) introducing said isolated members into a host cell population;
(d) performing a phenotypic assay with said population in order to identify a sublibrary of physiologically relevant perturbagens; and
(e) identifying individual protein-ligand pairs between members of said sublibrary of physiologically relevant perturbagens and said therapeutic target candidate, wherein an interaction of a physiologically relevant perturbagen with a therapeutic target candidate identifies said therapeutic target candidate as a physiologically relevant target molecule that correlates to a phenotype of interest.

3. The method of claims 1 or 2, wherein at least one of said protein-ligand interactions are determined by performing a yeast two-hybrid assay.

4. The method of claim 1 or claim 2, wherein said phenotypic assay is in high throughput format.

5. The method of claims 1 or 2, further comprising the step of using said physiologically relevant target molecule to screen for therapeutic compounds.

6. The method of claim 5, wherein said screen comprises testing for agents that disrupt the interaction between said physiologically relevant target molecule and its corresponding physiologically relevant perturbagen.

7. The method of claim 1, wherein said pool of therapeutic target candidates is encoded by an expression library.

8. The method of claim 7, wherein the expression library is genomic DNA.

9. The method of claim 7, wherein the expression library is cDNA.

10. The method of claims 1 or 2, wherein said putative perturbagen library is fused to a stabilizing polypeptide.

11. The method of claim 10, wherein said stabilizing polypeptide is a GFP.

12. The method of claim 3, further comprising the step of eliminating bait sequences that self-activate.

13. The method of claim 3, wherein the yeast two-hybrid system utilizes a GAL4-based reporter system.

14. The method of claim 3, wherein the yeast two-hybrid system utilizes LexA-based reporter system.

15. The method of claim 1 or claim 2, wherein said phenotypic assay is a direct assay.

16. The method of claim 15, wherein said direct assay detects changes in growth.

17. The method of claim 16, wherein said direct assay relates to cancer.

18. The method of claim 17, wherein said cancer is selected from a group consisting of melanoma, breast cancer, cervical cancer and colon cancer.

19. The method of claim 1 or claim 2, wherein said phenotypic assay is an indirect assay.

20. The method of claim 19, wherein said indirect assay relates to diabetes.

21. The method of claim 1 wherein said pool of therapeutic target candidates represents substantially all open reading frames of the human proteome.

22. The method of claim 1 wherein said pool of therapeutic target candidates represents a selected subset of the human proteome.

23. The method of claim 1 wherein said pool of therapeutic target candidates represents a family of related proteins.

24. The method of claim 23, wherein said family is selected from a group consisting of kinases, phosphatases, G-coupled protein receptors, kinases, phosphatases, secreted proteins, G proteins, cyclins, transcription factors and integrins.
